(19) United States
(12) Patent Application Publication
(10) Pub. No.: US 2024/0325152 A1
(43) Pub. Date: Oct. 3, 2024

(54) Methods and Devices for Improving Bone Healing

(71) Applicant: Colorado State University Research Foundation, Fort Collins, CO (US)

(72) Inventors: David Prawel, Loveland, CO (US); James Walter Johnson, Fort Collins, CO (US); Vail Olin Baumer, Fort Collins, CO (US)

(21) Appl. No.: 18/624,342

(22) Filed: Apr. 2, 2024

Related U.S. Application Data
(60) Provisional application No. 63/493,934, filed on Apr. 3, 2023.

(51) Int. Cl. A61F 2/28; A61F 2/30

(52) U.S. Cl. CPC A61F 2/2846; A61F 2002/2835; A61F 2002/285; A61F 2002/30738

(57) ABSTRACT

Various implementations include a method for treating a bone defect of a first bone structure and a second bone structure of a patient. The method includes positioning a biodegradable scaffold within a passage of a biodegradable sleeve, wherein the passage extends from a first end to a second end of the biodegradable sleeve, wherein a geometry of the biodegradable sleeve induces strain on a portion of the biodegradable scaffold when a force is applied to the biodegradable sleeve; and positioning the biodegradable sleeve along the first bone structure and the second bone structure to span the bone defect.

### BACKGROUND

Critical bone defects occur with unfortunately high frequency in both human veterinary patients, due to cancer and trauma. Poor healing of critical-sized bone defects remains one of the biggest challenges in human and veterinary orthopedic medicine, often resulting in clinical complications, reoperations, poor functional outcomes, and ultimately limb loss, causing significant clinical and economic cost. Researchers have worked on this problem for decades using a wide variety of bone regeneration techniques and biomaterials, often involving 3D printed scaffolds to enhance bone growth, also referred to as bioactivity (osteoconduction, osteoinduction, and osseointegration). Scaffolds made with biomaterials like hydroxyapatite (HAp), beta-tri-calcium phosphate (β-TCP), anorganic (deproteinized) bone mineral and numerous other forms of calcium phosphates and derivatives thereof are highly osteogenic but are too brittle to enable any practical load bearing. Contrarily, enhancing the load-bearing capability of these osteogenic materials results in scaffolds with reduced osteogenic character. To date, no one has overcome this paradox for critical, load-bearing bone defects. The current standard of care for limb-sparing uses permanent fixation with large metal plates and endoprostheses that remain in the patient for lifetime.

Eighty-five percent of all skeletal tumors in dogs are osteosarcomas (see Vail, D. M. et al. Withrow & MacEwen's Small Animal Clinical Oncology. Elsevier Saunders; 2013.), most often affecting the proximal humerus and distal radius. Limb-sparing procedures are considered the standard of care in humans. In dogs, limb sparing is indicated in dogs with significant concurrent orthopedic or neurologic disease but is also often performed because the owners are strongly opposed to amputation. These procedures involve ostectomy of the cancerous and surrounding tissue, typically from the diaphysis through and including the epiphysis of the bone, followed by implantation of a large locking fixation plate and endoprosthesis. See Mitchell K. E. et al. Outcomes of Limb-Sparing Surgery Using Two Generations of Metal Endoprosthesis in 45 Dogs With Distal Radial Osteosarcoma. A Veterinary Society of Surgical Oncology Retrospective Study: Limb Sparing Endoprosthesis for Canine Radial Osteosarcoma. Veterinary Surgery 2016; 45:36-43; Liptak J M et al. Cortical Allograft and Endoprosthesis for Limb-Sparing Surgery in Dogs with Distal Radial Osteosarcoma: A Prospective Clinical Comparison of Two Different Limb-Sparing Techniques. Vet Surgery 2006; 35:518-33. This approach presents complications such as screw loosening, plate fracture, infection, and significant metal remaining in the body for the remainder of the animal's life. These challenges are also experienced in human medicine. Poor healing of these critical-sized bone defects remains one of the biggest challenges in human and veterinary orthopedic surgery, often resulting in limb loss (see Liptak J M et al.), leading to poor long-term outcomes with complication rates as high as 48% in humans (see Vidal L et al. Reconstruction of Large Skeletal Defects: Current Clinical Therapeutic Strategies and Future Directions Using 3D Printing. Front Bioeng. Biotechnol. 2020; 8:61.) and over 90% in dogs (see Mitchell K E et al.), reoperations, and poor functional outcomes, resulting in significant negative clinical and economic impact. Other strategies such as autografts, allografts and xenografts also have major drawbacks, including shortage of available tissue, thus there is a critical need to address this challenge. Tissue engineering solutions have emerged that use synthetic scaffolds to provide structure for new growing bone. Countless materials have been considered for scaffold development.

Hydroxyapatite (HAp), beta-tri-calcium phosphate (β-TCP), and numerous other forms of calcium phosphates and derivatives thereof are widely studied for bone regeneration scaffolds. See Bose S et al. Additive manufacturing of biomaterials. Progress in Materials Science 2018; 93:45-111; Vondran et. al. 3D printing of ceramic implants. MRS Bulletin 2016; 41:71; Suwanprateeb J et al. Mechanical and in vitro performance of apatite-wollastonite glass ceramic reinforced hydroxyapatite composite fabricated by 3D-printing. J Mater Sci: Mater Med 2009; 20:1281. These materials are bioreplaceable by new, native bone. They release calcium during degradation, which supports bone formation, resulting in excellent osteoconductivity. See Vondran et al. Despite excellent bone regeneration properties, success of these scaffolds is hampered by inadequate structural strength and stiffness required for acceptable load-bearing. See Bose S et al. This is especially challenging in critical defects. Polycaprolactone (PCL) is widely used polymeric biomaterial (see Lu L et al. Biocompatibility and biodegradation studies of PCL/β-TCP bone tissue scaffold fabricated by structural porogen method. J Mater Sci: Mater Med 2012; 23:2217-26.) due to its excellent biocompatibility, and is FDA approved for medical use. PCL is popular in bone tissue engineering due to its slow degradation and high stiffness. See Eshraghi S et al. Mechanical and microstructural properties of polycaprolactone scaffolds with one-dimensional, two-dimensional, and three-dimensional orthogonally oriented porous architectures produced by selective laser sintering. Acta Biomaterialia 2010; 6:2467-76; Brunello G et al. Powder-based 3D printing for bone tissue engineering. Biotechnology Advances 2016; 34:740-53. PCL is also radiolucent, enabling real-time radiographic assessment. See Choi S et al. New clinical application of three-dimensional-printed polycaprolactone/β-tricalcium phosphate scaffold as an alternative to allograft bone for limb-sparing surgery in a dog with distal radial osteosarcoma. The Journal of Veterinary Medical Science 2019; 81:434-9.

3D printing (3DP) has emerged as a popular method to fabricate complex shaped structures with high precision. 3DP enables creation of patient-specific scaffolds directly from CT scans, ensuring the scaffolds precisely fit the defect site, improving outcomes because the morphology of each bone and the percentage of bone removed varies between patients, depending on the size of the tumor. See Harrysson OLA et al. Applications of Metal Additive Manufacturing in Veterinary Orthopedic Surgery n.d.: 8; Seguin B et al. Limb-sparing in dogs using patient-specific, three-dimensional-printed endoprosthesis for distal radial osteosarcoma: A pilot study. Vet Comp Oncol 2020; 18:92-104. Patient-specific implants allow the implants to fit as perfectly as possible for each patient thereby enabling loads to be transmitted through the limb. Interrelated parameters such as porosity, permeability, pore size, shear stress, bulk material, and shape (topology) dictate the success of a scaffold. See Montazerian H et al. Longitudinal and radial permeability analysis of additively manufactured porous scaffolds: Effect of pore shape and porosity. Materials & Design 2017; 122:146-56; Rodríguez-Montaño ÓL et al. Comparison of the mechanobiological performance of bone tissue scaffolds based on different unit cell geometries. Journal of the Mechanical Behavior of Biomedical Materials 2018; 83:28-45; Abueidda D W et al. Mechanical properties of 3D printed polymeric Gyroid cellular structures: Experimental and finite element study. Materials & Design 2019; 165:107597; Melchels FPW et al. Effects of the architecture of tissue engineering scaffolds on cell seeding and culturing. Acta Biomaterialia 2010; 6:4208-17. Advanced, structurally-optimized scaffold topologies such as Gyroids (see Montazerian H et al.; Melchels FPW et al. Mathematically defined tissue engineering scaffold architectures prepared by stereolithography. Biomaterials 2010; 31:6909-16.) are enabling 3D printing of stiffer, stronger scaffolds (see Rodríguez-Montaño ÓL et al.; Abueidda D W et al.; Yuan L et al. Additive manufacturing technology for porous metal implant applications and triple minimal surface structures: A review. Bioactive Materials 2019; 4:56-70.).

Recombinant human bone morphogenic protein-2 (rhBMP-2) is one of the most widely used growth factors in both human (see Lissenberg-Thunnissen S N et al. Use and efficacy of bone morphogenetic proteins in fracture healing. International Orthopaedics (SICOT) 2011; 35:1271-80.) and veterinary (see Pinel C B, Pluhar G E. Clinical application of recombinant human bone morphogenetic protein in cats and dogs: A review of 13 cases n.d.; 53:8; Boudrieau, R. Initial Experience With rhBMP-2 Delivered in a Compressive Resistant Matrix for Mandibular Reconstruction in 5 Dogs. Vet Surg 2015; 44:443-58.) orthopedic surgery because of its powerful osteoinductive properties. A suitable carrier is required for sustained and local delivery of rhBMP-2. See El Bialy I et al. Formulation, Delivery and Stability of Bone Morphogenetic Proteins for Effective Bone Regeneration. Pharm Res 2017; 34:1152-70. Various forms of calcium phosphate have proven successful in numerous BMP delivery systems for large defect bone healing. Despite some reports of hypertrophy observed in some experimental (see Minier K et al. BMP-2 delivered from a self-cross-linkable CaP/hydrogel construct promotes bone regeneration in a critical-size segmental defect model of non-union in dogs. Vet Comp Orthop Traumatol 2014; 27:411-21.) and clinical (see Pinel C B et al.; Boudrieau R et al.) cases treated with rhBMP-2, recent studies counter that RhBMP-2 can have anti-neoplastic effects against osteosarcoma (see Xiong Q et al. BMP-2 inhibits lung metastasis of osteosarcoma: an early investigation using an orthotopic model. OTT 2018; Volume 11:7543-53; Rici R E G et al. Combination therapy of canine osteosarcoma with canine bone marrow stem cells, bone morphogenetic protein and carboplatin in an in vivo model n.d.: 11; Grassi Rici R et al. Mesenchymal stem cells with rhBMP-2 inhibits the growth of canine osteosarcoma cells. BMC Vet Res 2012; 8:17.).

Although existing techniques for treating bone defects may be suitable in certain applications, there remains a need for improved methods and devices for treating bone defects, such as a critical-sized bone defects, of human or veterinary patients, which may overcome one or more of the drawbacks associated with existing techniques.

### SUMMARY

Various implementations include a method for treating a bone defect of a first bone structure and a second bone structure of a patient. The method includes positioning a biodegradable scaffold within a passage of a biodegradable sleeve, wherein the passage extends from a first end to a second end of the biodegradable sleeve, wherein a geometry of the biodegradable sleeve induces strain on a portion of the biodegradable scaffold when a force is applied to the biodegradable sleeve; and positioning the biodegradable sleeve along the first bone structure and the second bone structure to span the bone defect.

In some implementations, the biodegradable sleeve includes a first sleeve portion and a second sleeve portion. In some implementations, the first sleeve portion has a greater rigidity than the second sleeve portion. In some implementations, the first sleeve portion includes a material that is not included in the second sleeve portion. In some implementations, the first sleeve portion has a larger wall thickness than the second sleeve portion as measured from an outer surface of the sleeve to an inner surface of the sleeve. In some implementations, the second sleeve portion includes a concavity defined by the outer surface of the sleeve. In some implementations, the first sleeve portion includes one or more ribs extending outwardly from the outer surface of the sleeve.

In some implementations, the method further includes resecting a region of bone between the first bone structure and the second bone structure and encompassing the bone defect before positioning the biodegradable scaffold within the passage of the biodegradable sleeve.

In some implementations, the biodegradable scaffold includes a biodegradable osteogenic scaffold.

In some implementations, the method further includes coupling the biodegradable sleeve to a fixation member before positioning the biodegradable sleeve and attaching the fixation member to each of the first bone structure and the second bone structure after positioning the biodegradable sleeve. In some implementations, the method does not include coupling the biodegradable sleeve to a fixation member.

In some implementations, coupling the biodegradable sleeve to the fixation member comprises sliding the biodegradable sleeve onto the fixation member.

In some implementations, attaching the fixation member to each of the first bone structure and the second bone structure includes attaching the fixation member to the first bone structure using a first plurality of screws and attaching the fixation member to the second bone structure using a second plurality of screws.

In some implementations, the method further includes treating the biodegradable sleeve with an antibiotic to inhibit infection. In some implementations, the biodegradable sleeve includes embedded bioactive materials such as antibiotics, which can be released over time.

In some implementations, the method further includes detaching the fixation member from the first bone structure and the second bone structure and removing the fixation member from the patient after the bone defect has healed and the biodegradable scaffold and the biodegradable sleeve have degraded.

In some implementations, the method further includes treating the biodegradable scaffold with one or more bioactive agents to facilitate osteoinduction, osteoconduction, osteointegration, vascularization, or angiogenesis.

In some implementations, the method further includes treating the fixation member with an antibiotic to inhibit infection.

In some implementations, loading of the first bone structure or second bone structure causes the application of force to the biodegradable sleeve for the purposes including but not limited to introducing micromotion (e.g., into the scaffold), guiding bone remodeling or cell growth, etc. In some implementations, the first bone structure or second bone structure remain static relative to the biodegradable sleeve during loading of the first bone structure or second bone structure.

In some implementations, positioning the biodegradable sleeve between the first bone structure and the second bone structure includes inserting an end portion of the first bone structure into the first end of the biodegradable sleeve such that the end portion of the first bone structure contacts the biodegradable scaffold and inserting an end portion of the second bone structure into the second end of the biodegradable sleeve such that the end portion of the second bone structure contacts the biodegradable scaffold.

In some implementations, the method further includes obtaining computed tomography scans of the patient and fabricating the biodegradable scaffold and the biodegradable sleeve based at least in part on the computed tomography scans. In some implementations, the method further includes fabricating a cutting guide based at least in part on the computed tomography scans. In some implementations, resecting the region of bone between the first bone structure and the second bone structure and encompassing the bone defect includes resecting the region of bone using the cutting guide.

In some implementations, the biodegradable scaffold is formed of a calcium phosphate-based material (e.g., naturally-derived or synthetic) or a composite including a calcium phosphate and a polymer. In some implementations, the biodegradable sleeve is formed of polycaprolactone or a composite including a calcium phosphate and polycaprolactone.

Various other implementations include a device for treating a bone defect of a bone having a first bone structure and a second bone structure. The device includes a biodegradable scaffold and a biodegradable sleeve configured for positioning between the first bone structure and the second bone structure. In some implementations, the biodegradable sleeve defines a passage configured for accepting the biodegradable scaffold. In some implementations, the passage extends from a first end to a second end of the biodegradable sleeve. In some implementations, a geometry of the biodegradable sleeve induces strain on a portion of the biodegradable scaffold when a force is applied to the biodegradable sleeve.

In some implementations, the biodegradable sleeve includes a first sleeve portion and a second sleeve portion, wherein the first sleeve portion has a greater rigidity than the second sleeve portion. In some implementations, the first sleeve portion includes a material that is not included in the second sleeve portion. In some implementations, the first sleeve portion has a larger wall thickness than the second sleeve portion as measured from an outer surface of the sleeve to an inner surface of the sleeve. In some implementations, the second sleeve portion includes a concavity defined by the outer surface of the sleeve. In some implementations, the first sleeve portion includes one or more ribs extending outwardly from the outer surface of the sleeve.

In some implementations, the biodegradable scaffold includes a biodegradable osteogenic scaffold.

In some implementations, the device further includes a fixation member configured for coupling to the biodegradable sleeve and attaching to each of the first bone structure and the second bone structure.

In some implementations, the passage is a first passage extending from the first end to the second end of the biodegradable sleeve. In some implementations, the biodegradable sleeve includes a second passage extending from the first end to the second end of the biodegradable sleeve and configured for receiving a portion of the fixation member therein.

In some implementations, the biodegradable sleeve further includes support ribs disposed between the first passage and the second passage and extending from the first end to the second end of the biodegradable sleeve. In some implementations, the first passage is in communication with the second passage through a gap defined between the support ribs. In some implementations, a portion of the biodegradable scaffold extends into the gap when the biodegradable scaffold is positioned within the biodegradable sleeve.

In some implementations, the first passage is further configured for receiving an end portion of the first bone structure and an end portion of the second bone structure therein.

In some implementations, the biodegradable sleeve further includes a plurality of apertures disposed along one or more sides of the biodegradable sleeve and extending from an outer surface of the biodegradable sleeve to the first passage. In some implementations, the apertures are configured for controlling transcortical perfusion.

In some implementations, the biodegradable sleeve further includes a plurality of openings disposed along a side of the biodegradable sleeve and extending from an outer surface of the biodegradable sleeve to the second passage. In some implementations, the openings expose respective portions of the fixation member when the fixation member is coupled to the biodegradable sleeve and are configured for receiving respective collagen sponges carrying an antibiotic therein (or any other bioactive agent).

In some implementations, the biodegradable scaffold includes a scaffold passage extending from a first end to a second end of the biodegradable scaffold and configured for receiving a collagen sponge carrying bioactive agents. In some implementations, the scaffold passage is left empty. In some implementations, the biodegradable scaffold does not include a scaffold passage.

In some implementations, the fixation member includes a first portion configured for extending beyond the first end of the biodegradable sleeve when the fixation member is coupled to the biodegradable sleeve, a second portion configured for extending beyond the second end of the biodegradable sleeve when the fixation member is coupled to the biodegradable sleeve, and an intermediate portion configured for positioning within the second passage when the fixation member is coupled to the biodegradable sleeve.

In some implementations, the first end of the passage defines a first periosteum slot and the second end of the passage defines a second periosteum slot. In some implementations, the first periosteum slot is configured for insertion of an end portion of periosteum associated with the first bone structure and the second periosteum slot is configured for insertion of an end portion of periosteum associated with the second bone structure.

In some implementations, loading of the first bone structure or second bone structure causes the application of force to the biodegradable sleeve. In some implementations, the first bone structure or second bone structure remain static relative to the biodegradable sleeve during loading of the first bone structure or second bone structure.

In some implementations, the biodegradable scaffold is formed of a calcium phosphate-based material or a composite including a calcium phosphate and a polymer. In some implementations, the biodegradable sleeve is formed of polycaprolactone or a composite including a calcium phosphate and polycaprolactone.

### BRIEF DESCRIPTION OF DRAWINGS

Example features and implementations of the present disclosure are disclosed in the accompanying drawings. However, the present disclosure is not limited to the precise arrangements and instrumentalities shown. Similar elements in different implementations are designated using the same reference numerals.

FIG. 1A is a top view radiograph of a current standard of care for treating a critical bone defect in a limb-sparing procedure, showing a metal locking plate.

FIG. 1B is a side view radiograph of a current standard of care for treating a critical bone defect in a limb-sparing procedure, showing a metal locking plate, screws, and an endoprosthetic spacer.

FIG. 2A is an exploded perspective view of an example device for treating a critical, long-bone defect in accordance with embodiments of the disclosure, showing a biodegradable sleeve, a biodegradable osteogenic scaffold, a fixation member, and a plurality of biodegradable retention cuffs of the device.

FIG. 2B is a perspective view of the device of FIG. 2A, showing the biodegradable sleeve, the biodegradable osteogenic scaffold, the fixation member, and the biodegradable cuffs in an assembled state.

FIG. 2C is a detailed side view of a portion of the biodegradable osteogenic scaffold, showing a gyroid topology of the scaffold.

FIG. 3A is a perspective view of an example device for treating a bone defect in accordance with embodiments of the disclosure, showing a biodegradable sleeve, a fixation member, and a plurality of screws of the device in relation to a proximal bone structure and a distal bone structure in accordance with an example use of the device.

FIG. 3B is a perspective view of the device of FIG. 3A, showing the biodegradable sleeve, the fixation member, and the screws in relation to the proximal bone structure and the distal bone structure.

FIG. 3C is a perspective view of a portion of the device of FIG. 3A, showing the fixation member coupled to the biodegradable sleeve.

FIG. 3D is a perspective view of a portion of the device of FIG. 3A, showing the fixation member coupled to the biodegradable sleeve.

FIG. 3E is a perspective view of a portion of the device of FIG. 3A, showing a biodegradable osteogenic scaffold of the device positioned within the biodegradable sleeve.

FIG. 3F is a perspective view of a portion of the device of FIG. 3A, showing the fixation member coupled to the biodegradable sleeve and the biodegradable osteogenic scaffold positioned within the biodegradable sleeve.

FIG. 4 is a perspective view of a biodegradable sleeve, according to another implementation.

FIG. 5A is a cross-sectional perspective view of a device with the sleeve of FIG. 4, with the host bone visible, according to another implementation.

FIG. 5B is a perspective view of the device with the sleeve of FIG. 4.

FIG. 6 is a side view radiograph of the device of FIGS. 5A and 5B for treating a critical bone defect in a limb-sparing procedure.

FIG. 7 is a perspective view of a biodegradable sleeve and biodegradable scaffold for treating a mandibular bone defect, according to another implementation.

FIG. 8 is a perspective view of a biodegradable sleeve and biodegradable scaffold for treating a mandibular bone defect, according to another implementation.

### DETAILED DESCRIPTION

In the following description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional. In some instances, well known methods, procedures, and/or components have not been described in detail so as not to unnecessarily obscure aspects of the embodiments.

### Overview

Embodiments of methods and devices for treating a bone defect, such as a critical-sized bone defect, extending between a proximal bone structure and a distal bone structure of a patient are provided. Some implementations provide for methods and devices for treating a non-segmental bone defect. As described herein, example methods generally may include resecting a region of bone between the proximal bone structure and the distal bone structure and encompassing the bone defect, positioning a biodegradable osteogenic scaffold within a biodegradable sleeve, coupling the biodegradable sleeve to an optional fixation member, positioning the biodegradable sleeve between or along the proximal bone structure and the distal bone structure, and, optionally, attaching the fixation member to each of the proximal bone structure and the distal bone structure. In this manner, the fixation member may provide fixation of the proximal bone structure and the distal bone structure across the bone defect to enable normal load bearing, the biodegradable osteogenic scaffold may facilitate new bone growth between the proximal bone structure and the distal bone structure, and the biodegradable sleeve may protect the biodegradable osteogenic scaffold while new bone grows and also contain the new bone growth. Over time, the biodegradable osteogenic scaffold and the biodegradable sleeve may degrade, the new, natural bone growth may provide complete bone healing between the proximal bone structure and the distal bone structure, and the fixation member may be removed from the patient, leaving only endogenous bone. As described herein, example devices generally may include a biodegradable sleeve configured for positioning between the proximal bone structure and the distal bone structure, a biodegradable osteogenic scaffold configured for positioning within the biodegradable sleeve, and a fixation member configured for coupling to the biodegradable sleeve and attaching to each of the proximal bone structure and the distal bone structure. Various configurations of the device components may be used in different embodiments.

The present disclosure provides endoprosthetic designs and surgical procedures that can be used in veterinary and human orthopedic medicine. As discussed above, HAp, (3-TCP, anorganic (deproteinized) bone mineral, and numerous other forms of calcium phosphates and derivatives thereof are excellent and widely used biomaterials for bone regeneration but provide insufficient mechanical support for acceptable load bearing. Many researchers are succeeding at strengthening scaffolds comprising biomaterials using a wide variety of additives and infiltration techniques, but thus far at the cost of reductions in bioactivity. The methods and devices described herein can leverage the fixation member to support the load, as it already functions in the current standard of care, and as a means to connect to and protect an osteogenic calcium-phosphate-based scaffold so that the scaffold can enable new bone growth and bioactivity. A critical-sized, patient-specific, biodegradable endoprostheses may be 3D printed to protect the scaffold and augment its poor mechanical properties, while working alongside the fixation member. The scaffold may be treated with rhBMP-2 to enhance bone healing, as recently demonstrated for large bone defects (see Franch J et al. Franch J, Barba A, Rappe K, Maazouz Y, Ginebra M. Use of three-dimensionally printed β-tricalcium phosphate synthetic bone graft combined with recombinant human bone morphogenic protein-2 to treat a severe radial atrophic nonunion in a Yorkshire terrier. Veterinary Surgery 2020: vsu.13476. https://doi.org/10.1111/vsu.13476. Henkel J, Medeiros Savi F, Berner A, Fountain S, Saifzadeh S, Steck R, et al. Scaffold-guided bone regeneration in large volume tibial segmental defects. Bone 2021; 153:116163. https://doi.org/10.1016/j.bone.2021.116163. Yang Y P, Labus K M, Gadomski B C, Bruyas A, Easley J, Nelson B, et al. Osteoinductive 3D printed scaffold healed 5 cm segmental bone defects in the ovine metatarsus. Sci Rep 2021; 11:6704. https://doi.org/10.1038/s41598-021-86210-5. Use of three-dimensionally printed β-TCP synthetic bone graft combined with recombinant human bone morphogenic protein-2 to treat a severe radial atrophic nonunion in a Yorkshire terrier. Veterinary Surgery 2020: vsu.13476.). As described, the present techniques in limb sparing treatment of a bone defect, based on current standard of care, use bioreplaceable endoprostheses to enable a highly osteogenic scaffold to support healthy bone growth under normal loads. Ultimately, the techniques can enable complete bone healing and removal of fixation, leaving the patient with endogenous bone as in the Franch study (see Franch J et al.), after the endoprostheses degrades safely in the body.

Although initial development of the disclosed methods and devices has been performed in dogs, the methods and devices translate to human medicine fairly easily. The dog is a highly suitable model for human bone from a biological standpoint (see Bae J et al. Development and Assessment of a 3D-Printed Scaffold with rhBMP-2 for an Implant Surgical Guide Stent and Bone Graft Material: A Pilot Animal Study. Materials 2017; 10:1434.), with similarities in microstructure and remodeling. Large dogs weigh as much as a small to mid-sized human. Furthermore, the techniques described herein depend on structural design improvements rather than complex physiological or biological approaches, that translate more readily to human medicine. Medical devices for canine orthopedics are often identical to those for humans, making possible translation of devices. Materials used for the scaffold and endoprostheses have been thoroughly studied and known to be biocompatible for implantation in live animals and FDA approved for many applications. The disclosed methods and devices can significantly improve patient outcomes in limb-sparing procedures, for animals and humans alike.

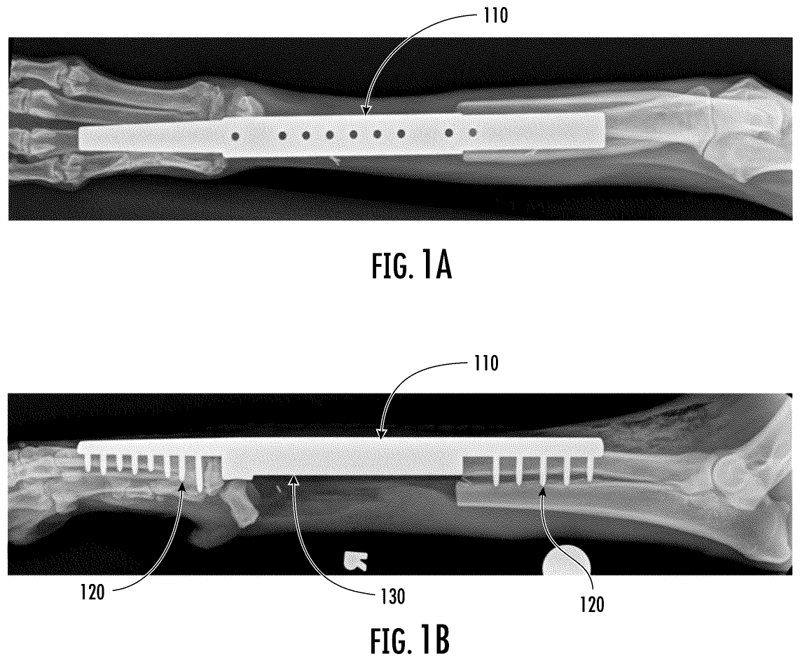

FIGS. 1A and 1B illustrate one instance of a standard of care for treating a bone defect, specifically an epiphyseal defect in a limb of a canine patient being treated for osteosarcoma. In this case, as shown, a distal portion of the patient's radius encompassing the tumor may be removed, along with a distal portion of the patient's ulna. Fixation of the limb may be provided by a metal locking plate 110 that is attached via screws 120 to each of a proximal bone structure and a distal bone structure. As shown, a proximal end portion of the locking plate 110 may be attached to the remaining host radius, and a distal end portion of the locking plate 110 may be attached to the third metacarpal. An endoprosthesis 130 may be positioned between the proximal bone structure and the distal bone structure to act as a spacer for limiting movement of the proximal bone structure relative to the distal bone structure to inhibit loosening of the locking plate 110. As shown, the endoprosthesis 130 may be positioned between the remaining host radius and the radial carpus. Although the depicted example shows the locking plate 110, the screws 120, and the endoprosthesis 130 being used to treat an epiphyseal defect, these components similarly may be used to treat a segmental defect in a limb of a patient. As discussed above, the standard of care approach may present certain complications, including screw loosening, plate fracture, infection, and significant metal remaining in the body for the remainder of the patient's life.

### Example Devices and Methods for Treating a Bone Defect

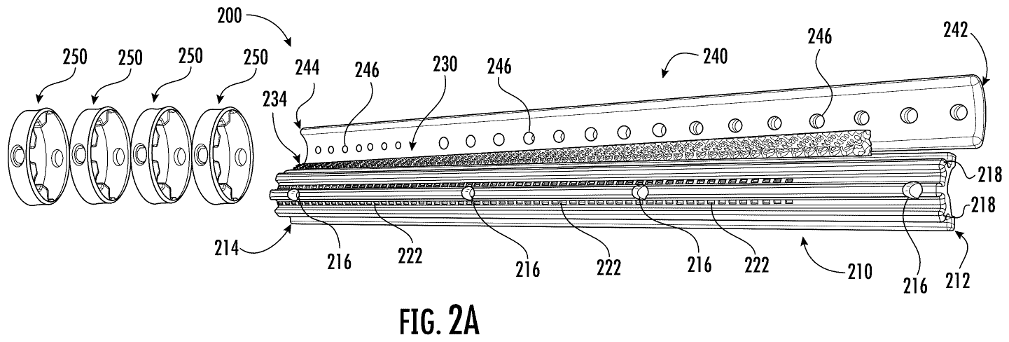

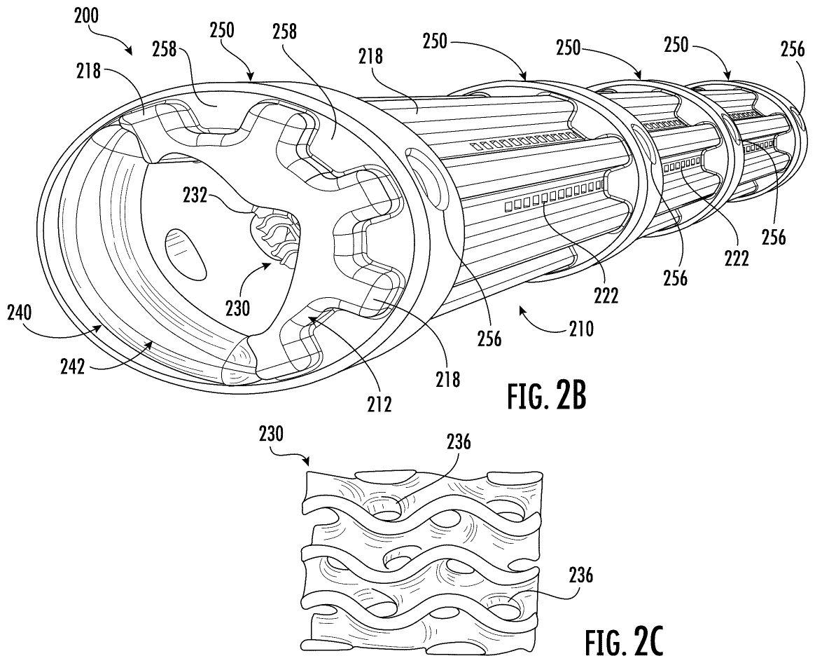

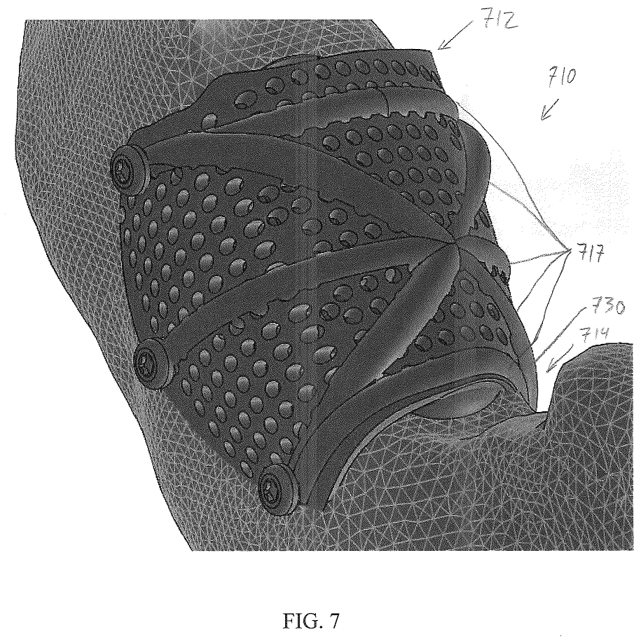

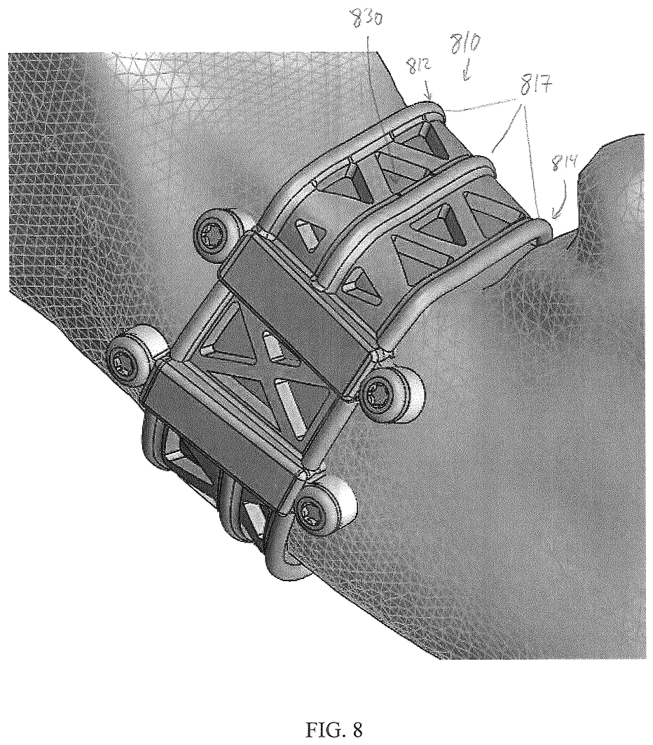

FIGS. 2A-2C illustrate an example device 200 for treating a bone defect, such as a critical-sized bone defect, of a veterinary or human patient. In some instances, the device 200 may be used to treat an epiphyseal defect. In other instances, the device 200 may be used to treat a segmental defect, a mandibular defect (as shown in FIGS. 7 and 8), or other type of bone defect. As shown, the device 200 may include a biodegradable sleeve 210, a biodegradable osteogenic scaffold 230, a fixation member 240, and a plurality of biodegradable cuffs 250. It will be appreciated that the size and shape of the respective components of the device 200 may be varied to accommodate a particular patient and the particular defect being treated. As discussed below, in some embodiments, the biodegradable sleeve 210, the biodegradable osteogenic scaffold 230, and the biodegradable cuffs 250 may be fabricated as patient-specific components. For example, these components may be 3D printed based on CT scans of the patient's limb. In some embodiments, the fixation member 240 may be a commercially-available component used in accordance with the standard of care, with the size of the fixation member 240 being selected to accommodate the patient and defect to be treated.

As shown, the biodegradable sleeve 210 may be formed as an elongate structure having a proximal end 212 and a distal end 214 disposed opposite one another along the longitudinal axis of the biodegradable sleeve 210. In some embodiments, as shown, the proximal end 212 may be shape-matched to a distal portion of the proximal bone structure of the patient, and the distal end 214 may be shape matched to a proximal portion of the distal bone structure of the patient. For example, for an epiphyseal defect, the proximal end 212 may be shape-matched to the remaining host radius, and the distal end 214 may be shape-matched to the radial carpus. As used herein, the term “sleeve” can include any shape structure that defines a passage, such as any closed cross-sectional shape or open cross-sectional shape. For example, in some embodiments, such as the biodegradable sleeves 210, 710 shown in FIGS. 2A-2C and 7, the biodegradable sleeve 210, 710 may have a generally C-shaped cross-sectional shape (i.e., taken perpendicular to the longitudinal axis of the biodegradable sleeve 210, 710), with a gap extending along one side of the biodegradable sleeve 210, 710. In some implementations, such as the biodegradable sleeve 810 shown in FIG. 8, the biodegradable sleeve 810 can include two C-shaped cross-sectional halves that are couplable to each other to form a “clamshell” style sleeve that can be applied around a bone defect. In this manner, the biodegradable sleeve 210 may include a passage configured for receiving the biodegradable osteogenic scaffold 230 therein during use of the device 200. In some embodiments, as shown, the biodegradable sleeve 210 may include a plurality of screw holes 216 extending therethrough and spaced apart from one another along the length of the biodegradable sleeve 210. As discussed below, the screw holes 216 may facilitate connection of the biodegradable sleeve 210 to the fixation member 240 and the biodegradable cuffs 250. In some embodiments, as shown, the biodegradable sleeve 210 may include a plurality of ribs 218 each extending from the proximal end 212 to the distal end 214. As discussed below, the ribs 218 may facilitate connection of the biodegradable sleeve 210 to the biodegradable cuffs 250 and load sharing therebetween. The ribs 218 can also be used to define two or more portions of the sleeve 210 having different wall thicknesses. The different thicknesses of the wall of the sleeve 210 have different rigidity enabling the geometry of the biodegradable sleeve 210 to induce strain on a portion of the biodegradable scaffold 230 when a force is applied to the biodegradable sleeve 210, as discussed in more detail below. In some embodiments, as shown, the biodegradable sleeve 210 may include a plurality of apertures 222 extending therethrough and spaced apart from one another along the length of the biodegradable sleeve 210. The apertures 222 may be configured for controlling transcortical perfusion during use of the device 200. In some embodiments, the apertures 222 may be omitted to prevent transcortical perfusion through the biodegradable sleeve 210. In some embodiments, the biodegradable sleeve 210 may be formed of polycaprolactone or a composite comprising a calcium phosphate and polycaprolactone. Other suitable biocompatible and biodegradable materials may be used for the biodegradable sleeve 210 in other embodiments.

The biodegradable osteogenic scaffold 230 may be formed as an elongate structure having a proximal end 232 and a distal end 234 disposed opposite one another along the longitudinal axis of the biodegradable osteogenic scaffold 230. In some embodiments, as shown, the biodegradable osteogenic scaffold 230 may have a gyroid topology, with a plurality of interconnected pores 236. Other porous topologies of the biodegradable osteogenic scaffold 230 may be used in other embodiments. In some embodiments, the biodegradable osteogenic scaffold 230 may have a porosity of 70%. In some embodiments, the biodegradable osteogenic scaffold 230 may include a hollow canal configured for allowing intracortical perfusion. As shown, the length of the biodegradable osteogenic scaffold 230 may be less than the length of the biodegradable sleeve 210. In this manner, the biodegradable osteogenic scaffold 230 may be positioned entirely within the biodegradable sleeve 210 during use of the device 200. In some embodiments, the biodegradable osteogenic scaffold 230 may be formed of a calcium phosphate, such as HAp or 3-TCP. In some embodiments, the biodegradable osteogenic scaffold 230 may be formed of a calcium phosphate-based material, anorganic (deproteinized) bone mineral, or a composite comprising a calcium phosphate and a polymer. Other suitable biocompatible, biodegradable, and osteogenic materials may be used for the biodegradable osteogenic scaffold 230 in other embodiments.

The fixation member 240 may be formed as an elongate structure having a proximal end 242 and a distal end 244 disposed opposite one another along the longitudinal axis of the fixation member 240. According to various embodiments, the fixation member 240 may be a fixation plate, a fixation rod, a locking plate, a locking rod, mandibular staple plate, or other type of suitable structure for fixation of bone structures. In some embodiments, as shown, the fixation member 240 may have a curved cross-sectional shape (i.e., taken perpendicular to the longitudinal axis of the fixation member 240). As discussed below, the fixation member 240 may be coupled to the biodegradable sleeve 210 such that the fixation member 240 covers the open side of the biodegradable sleeve 210, thereby containing the biodegradable osteogenic scaffold 230 between the fixation member 240 and the biodegradable sleeve 210. In some embodiments, as shown, the fixation member 240 may be inserted into the gap of the biocompatible sleeve 210. As shown, the fixation member 240 may include a plurality of screw holes 246 extending therethrough and spaced apart from one another along the length of the fixation member 240. As discussed below, some of the screw holes 246 may be used to facilitate connection of the fixation member 240 to the biodegradable sleeve 210 and the biodegradable cuffs 250, while other screw holes 246 may be used to facilitate connection of the fixation member 240 to a proximal bone structure and a distal bone structure during use of the device 200. The fixation member 240 may be formed of any suitable biocompatible, non-biodegradable material such that the fixation member 240 may be removed from the patient after the bone defect has healed and the biodegradable sleeve 210 and the biodegradable osteogenic scaffold 230 have degraded. According to various embodiments, the fixation member 240 may be formed of a metal, a polymeric material, a ceramic material, a composite material, or other suitable material for fixation of bone structures.

As shown, each of the biodegradable cuffs 250 may be formed as ring-shaped members configured for sliding over the biodegradable sleeve 210 and the fixation member 240. In some embodiments, as shown, edges of the biodegradable sleeve 210 and the fixation member 240 may be rounded to ease sliding of the biodegradable cuffs 250 onto the biodegradable sleeve 210 and the fixation member 240 as well as to minimize tissue trauma. In some embodiments, as shown, each of the biodegradable cuffs 250 may include a pair of screw holes 256 extending therethrough and disposed opposite one another. The screw holes 256 may facilitate connection of each of the biodegradable cuffs 250 to the biodegradable sleeve 210 and the fixation member 240. In some embodiments, as shown, for each of the biodegradable cuffs 250, a screw may be inserted through one of the screw holes 256 of the biodegradable cuff 250, through one of the screw holes 246 of the fixation member 240, through the biocompatible osteogenic scaffold 230, through one of the screw holes 216 of the biodegradable sleeve 210, and through the other screw hole 256 of the biodegradable cuff 250. This configuration may distribute load through the biodegradable cuff 250 into the fixation member 240, which may maintain the integrity and minimize mobility of the biocompatible osteogenic scaffold 230 as new bone develops. In some embodiments, as shown, each of the biodegradable cuffs 250 may include a plurality of protrusions 258 extending radially inward and configured for mating with the ribs 218 of the biodegradable sleeve 210. As shown, the mating engagement between the protrusions 258 and the ribs 218 may facilitate alignment of the screw holes 256 of the biodegradable cuff 250 with the screw holes 216 of the biodegradable sleeve 210 and the screw holes 246 of the fixation member 240. In some embodiments, the biodegradable cuffs 250 may be formed of polycaprolactone or a composite comprising a calcium phosphate and polycaprolactone. Other suitable biocompatible and biodegradable materials may be used for the biodegradable cuffs 250 in other embodiments.

As noted above, the biodegradable sleeve 210, the biodegradable osteogenic scaffold 230, and the biodegradable cuffs 250 may be fabricated as patient-specific components. In some embodiments, CT scans of the patient's limb may be used to fabricate the biodegradable sleeve 210, the biodegradable osteogenic scaffold 230, and the biodegradable cuffs 250 for the patient. In some embodiments, a series of two-dimensional DICOM images of the full extent of the limb may be imported (e.g., using In Vesalius) and segmented to create high-resolution digital models of the radius, ulna, manus, and metacarpals of the patient. The images then may be segmented based on Hounsfield attenuation values, and surface representations of the bone may be generated and then imported (e.g., into Fusion 360) where 3D models of the components may be created for 3D printing. In some embodiments, finite element analysis (FEA) may be used to investigate the ideal design parameters of the biodegradable sleeve 210 and the biodegradable cuffs 250 and the overall device 200, with and without the fixation member 240, to determine optimal geometry of the components and features thereof. Parameters such as rib height and thickness and mechanical properties such as structural modulus may be simulated to optimize the design of the device 200 prior to fabrication. In some embodiments, the CT scans also may be used to fabricate cutting guides to assure accurate surgical resections to match the geometry of the device 200.

The biodegradable osteogenic scaffold 230 may be 3D printed using existing methods. See Lopez Ambrosio, Katherine V. HYDROXYAPATITE STRUCTURES CREATED BY ADDITIVE MANUFACTURING WITH EXTRUDED PHOTOPOLYMER. Master's Thesis, Ft. Collins, CO: Colorado State University; 2019; 2019; Isaacson, Nelson, Katherine V. Lopez-Ambrosio, Laura Chubb, Nathan Waanders, Emily Hoffmann, Connor Witt, Susan P. James, David A. Prawel, Compressive Properties and Failure Behavior of Photocast Hydroxyapatite Gyroid Scaffolds Vary with Porosity, Journal of Biomaterials Applications, 2022, March, 1-22. DOI:10.1177/08853282211073904. In some embodiments, a photopolymer comprised of ethylene glycol dimethacrylate (EDGMA) and other polymers may be mixed with hydroxyapatite (HAp) particles into a shear-thinning slurry using a planetary ball mill. Slurries of 41 vol % HAp may be transferred to a syringe and 3D printed on 3D printer, such as a HyRel Engine S R printer with a 0.413 mm nozzle inner diameter, using a combination of viscous extrusion and photopolymer processes. The photopolymerization reaction may be initiated by continuous exposure of the deposited roads to a near-UV light source (405 nm wavelength), causing polymerization and hardening of the continuous phase, layer-by-layer. The sintered biodegradable osteogenic scaffold 230 may be non-cytotoxic. After printing, the biodegradable osteogenic scaffold 230 may undergo a two-step sintering process to eliminate the polymeric content and consolidate the HAp particles. In some embodiments, the printed biodegradable osteogenic scaffold 230 may be heated at 5° C./min up to 500° C. and held for 1 h, and then sintered at a heating rate of 15° C./min, up to 1150° C. and held for 5 h. This process may confer the sintered HAp structures with suitable mechanical properties. In some embodiments, the biodegradable sleeve 210 and the biodegradable cuffs 250 may be fabricated using a thermal process on a melt extrusion 3D printer (e.g., Prusa i3-MK3, Budapest, Hungary). PCL filament may be extruded by regulated ram pressure through a Flexion head. The extruded PCL may cool on a build plate to fabricate the components, which then may cool to room temperature. Prints may be oriented to maximize part strength and minimize support material.

The device 200 may be used to treat a bone defect, such as a critical-sized bone defect, extending between a proximal bone structure and a distal bone structure of a patient. In some embodiments, the bone defect may be an epiphyseal defect. In some embodiments, the bone defect may be a segmental defect. A method of using the device 200 for treating the bone defect generally may include resecting a region of bone between the proximal bone structure and the distal bone structure and encompassing the bone defect, positioning the biodegradable osteogenic scaffold 230 within the biodegradable sleeve 210, coupling the biodegradable sleeve 210 to the fixation member 240, positioning the biodegradable sleeve 210 between the proximal bone structure and the distal bone structure, and attaching the fixation member 240 to each of the proximal bone structure and the distal bone structure.

In some embodiments, the region of bone may be resected using one or more cutting guides fabricated based on CT scans of the patient's limb. For an epiphyseal defect, approximately 40% of the distal radius may be removed, along with a slightly larger section of the ulna to enable insertion of the device 200. In some embodiments, the biodegradable osteogenic scaffold 230 may be treated with one or more bioactive agents to facilitate bone growth or angiogenesis before positioning the biodegradable osteogenic scaffold 230 within the biodegradable sleeve 210. For example, the biodegradable osteogenic scaffold 230 may be treated with rhBMP-2 to facilitate bone growth and/or vascular endothelial growth factor (VEGF) to facilitate angiogenesis. In some embodiments, the biodegradable osteogenic scaffold 230 may be positioned within the biodegradable sleeve 210 such that the proximal end 232 of the biodegradable osteogenic scaffold 230 is offset from the proximal end 212 of the biodegradable sleeve 210, and a distal portion of the proximal bone structure may be inserted into the biodegradable sleeve 210 and in contact with the proximal end 232 of the biodegradable osteogenic scaffold 230. For an epiphyseal defect, a distal portion of the remaining host radius may be inserted into the biodegradable sleeve 210 and in contact with the proximal end 232 of the biodegradable osteogenic scaffold 230. In some embodiments, the biodegradable osteogenic scaffold 230 may be positioned within the biodegradable sleeve 210 such that the distal end 234 of the biodegradable osteogenic scaffold 230 is flush with the distal end 214 of the biodegradable sleeve 210, and a proximal portion of the distal bone structure may be positioned in close contact with the distal end 234 of the biodegradable osteogenic scaffold 230 and the distal end 214 of the biodegradable sleeve 210. For an epiphyseal defect, the proximal end of the carpus may be positioned in close contact with the distal end 234 of the biodegradable osteogenic scaffold 230 and the distal end 214 of the biodegradable sleeve 210. In some embodiments, the biodegradable osteogenic scaffold 230 may be positioned within the biodegradable sleeve 210 such that the distal end 234 of the biodegradable osteogenic scaffold 230 is offset from the distal end 214 of the biodegradable sleeve 210, and a proximal portion of the distal bone structure may be inserted into the biodegradable sleeve 210 and in contact with the distal end 234 of the biodegradable osteogenic scaffold 230.

In some embodiments, the biodegradable sleeve 210 may be coupled to the fixation member 240 before the biodegradable sleeve 210 is positioned between the proximal bone structure and the distal bone structure. In some embodiments, the biodegradable sleeve 210 may be coupled to the fixation member 240 by inserting the fixation member 240 into the gap of the biodegradable sleeve 210. In some embodiments, a proximal end portion of the fixation member 240 may be attached to the proximal bone structure using a first plurality of screws, and a distal end portion of the fixation member 240 may be attached to the distal bone structure using a second plurality of screws. For an epiphyseal defect, the proximal end portion of the fixation member 240 may be attached to the remaining host radius, and the distal end portion of the fixation member 240 may be attached to the third metacarpal.

In some embodiments, the biodegradable cuffs 250 may be slid over the biodegradable sleeve 210 and the fixation member 240 after the fixation member 240 is attached to the proximal bone structure but before the fixation member 240 is attached to the distal bone structure. One of the biodegradable cuffs 250 may be slid over the biodegradable sleeve 210 and the fixation member 240 from the distal ends 214, 244 toward the proximal ends 212, 242 thereof, aligned with the proximal-most screw hole 216 of the biodegradable sleeve 210, and connected to the biodegradable sleeve 210 and the fixation member 240 using a screw. The screw may pass through the biodegradable cuff 250, the fixation member 240, and the bone cortex of the proximal bone structure on the cis side and through the bone cortex of the proximal bone structure, the biodegradable sleeve 210, and the biodegradable cuff 250 on the trans side. In this manner, the screw may lock the proximal end of the device 200 into position. The screw should not protrude exceedingly far from the biodegradable cuff 250 to minimize risk of collateral tissue trauma. Each of the remaining biodegradable cuffs 250 then may be slid over the biodegradable sleeve 210 and the fixation member 240 from the distal ends 214, 244 toward the proximal ends 212, 242 thereof, aligned with one of the remaining screw holes 216 of the biodegradable sleeve 210, and connected to the biodegradable sleeve 210 and the fixation member 240 using a respective screw. Each of these screws may pass through the biodegradable cuff 250, the fixation member 240, the biodegradable osteogenic scaffold 230, the biodegradable sleeve 210, and the biodegradable cuff 250. After connecting the biodegradable cuffs 250, the distal end 214 of the biodegradable sleeve 210 and the distal end of the biodegradable osteogenic scaffold 230 may be positioned in close contact with a proximal portion of the distal bone structure, and the distal end portion of the fixation member 240 may be attached to the distal bone structure. For an epiphyseal defect, the distal end 214 of the biodegradable sleeve 210 and the distal end of the biodegradable osteogenic scaffold 230 may be positioned in close contact with the proximal end of the carpus, and the distal end portion of the fixation member 240 may be attached to the third metacarpal.

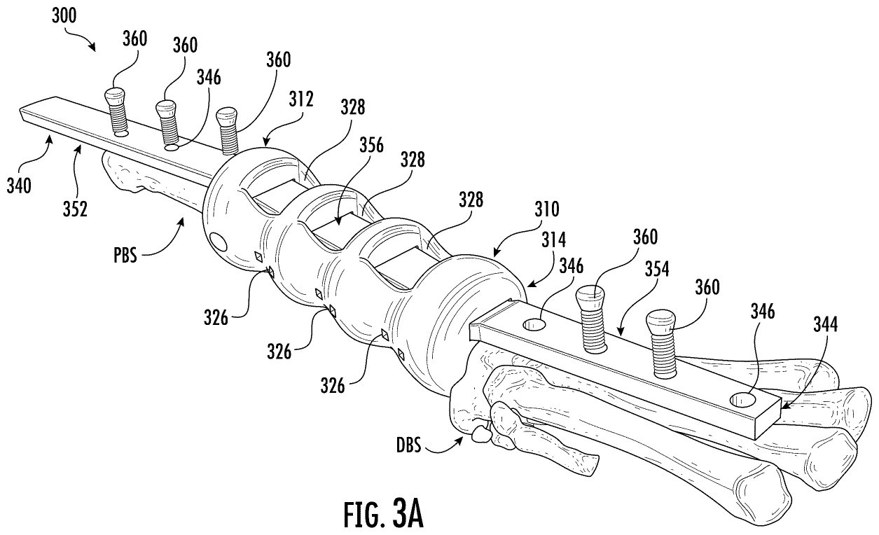

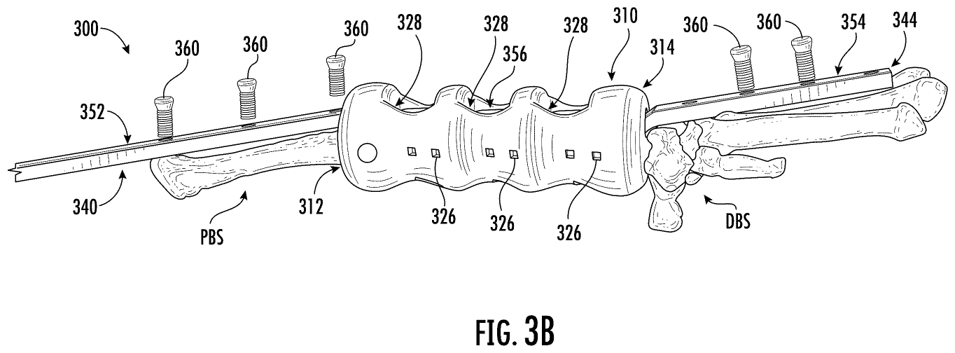

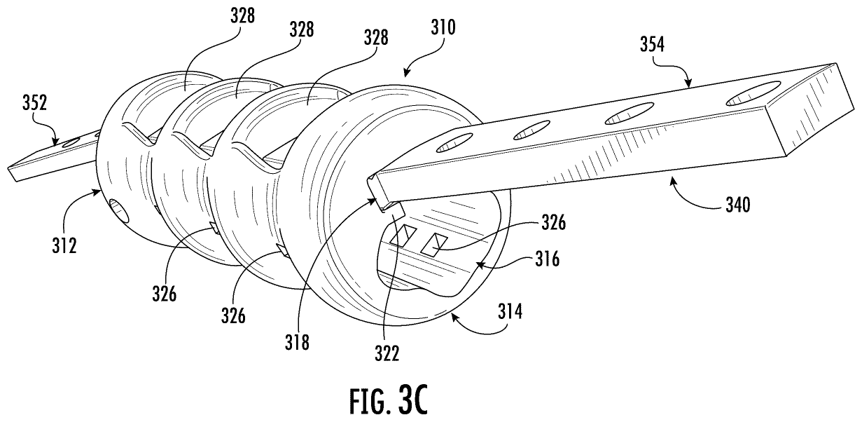

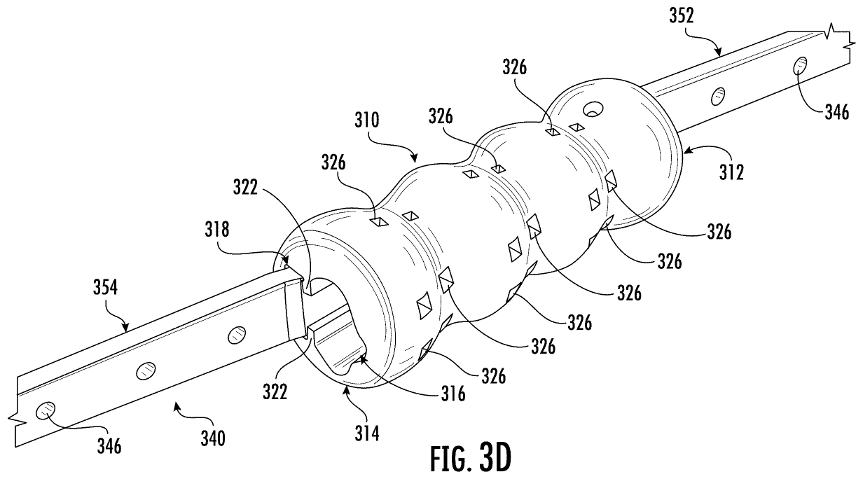

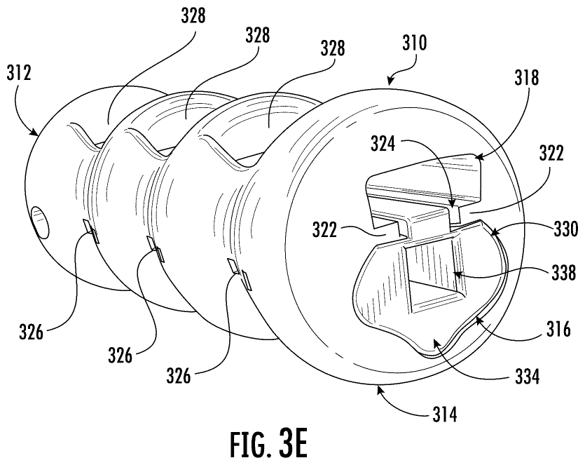

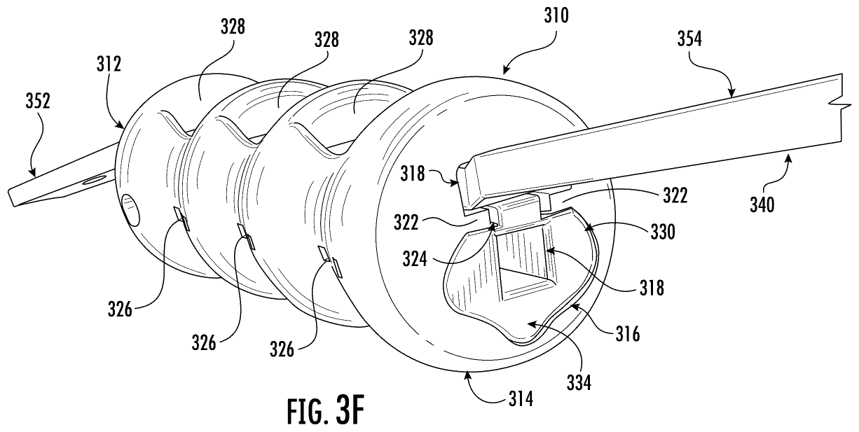

FIGS. 3A-3F illustrate another example device 300 for treating a bone defect, such as a critical-sized bone defect, of a veterinary or human patient. In some instances, the device 300 may be used to treat an epiphyseal defect. In other instances, the device 300 may be used to treat a segmental defect or a mandibular defect, as shown in FIGS. 7 and 8. Because components of the device 300 may be 3D printed according to patient anatomy, they can be appropriately configured to any surgical case. FIGS. 3A and 3B show the device 300 positioned relative to a proximal bone structure PBS and a distal bone structure DBS for treating an epiphyseal defect. As shown, the device 300 may include a biodegradable sleeve 310, a biodegradable osteogenic scaffold 330, a fixation member 340, and a plurality of screws 360. It will be appreciated that the size and shape of the respective components of the device 300 may be varied to accommodate a particular patient and the particular defect being treated. As discussed below, in some embodiments, the biodegradable sleeve 310 and the biodegradable osteogenic scaffold 330 may be fabricated as patient-specific components. For example, these components may be 3D printed based on CT scans of the patient's limb. In some embodiments, the fixation member 340 may be a commercially-available component used in accordance with the standard of care, with the size of the fixation member 340 being selected to accommodate the patient and defect to be treated.

As shown, the biodegradable sleeve 310 may be formed as an elongate structure having a proximal end 312 and a distal end 314 disposed opposite one another along the longitudinal axis of the biodegradable sleeve 310. In some embodiments, as shown, the proximal end 312 may be shape-matched to a distal portion of the proximal bone structure PBS of the patient, and the distal end 314 may be shape matched to a proximal portion of the distal bone structure DBS of the patient. For example, for an epiphyseal defect, the proximal end 312 may be shape-matched to the remaining host radius, and the distal end 314 may be shape-matched to the radial carpus. In some embodiments, as shown, the biodegradable sleeve 310 may have a generally circular cross-sectional shape (i.e., taken perpendicular to the longitudinal axis of the biodegradable sleeve 310). The biodegradable sleeve 310 may include a first passage 316 extending from the proximal end 312 to the distal end 314 and configured for receiving the biodegradable osteogenic scaffold 330 therein. As shown, the shape and size of the first passage 316 may correspond to the shape and size of the biodegradable osteogenic scaffold 330. The biodegradable sleeve 310 also may include a second passage 318 extending from the proximal end 312 to the distal end 314 and configured for receiving a portion of the fixation member 340 therein. As shown, the shape and size of the second passage 318 may correspond to the shape and size of the fixation member 340. In some embodiments, as shown, the biodegradable sleeve 310 may include a pair of support ribs 322 disposed between the first passage 316 and the second passage 318 and extending from the proximal end 312 to the distal end 314 of the biodegradable sleeve 310. As shown, a gap 324 may be defined between the ribs 322 such that the first passage 316 is in communication with the second passage 318 through the gap 324. In some embodiments, as shown, the biodegradable sleeve 310 may include a plurality of apertures 326 disposed along one or more sides of the biodegradable sleeve 310 and extending from an outer surface of the biodegradable sleeve 310 to the first passage 316. In this manner, the apertures 326 may be configured for controlling transcortical perfusion during use of the device 300. In some embodiments, the apertures 326 may be omitted to prevent transcortical perfusion through the biodegradable sleeve 310. In some embodiments, as shown, the biodegradable sleeve 310 may include a plurality of openings 328 disposed along a side of the biodegradable sleeve 310 and extending from an outer surface of the biodegradable sleeve 310 to the second passage 318. The openings 328 may expose respective portions of the fixation member 340 when the fixation member 340 is coupled to the biodegradable sleeve 310 and may be configured for receiving respective collagen sponges carrying one or more bioactive agents therein, such as an antibiotic to inhibit infection, during use of the device 300. In some embodiments, the biodegradable sleeve 310 may be formed of polycaprolactone or a composite comprising a calcium phosphate and polycaprolactone. Other suitable biocompatible and biodegradable materials may be used for the biodegradable sleeve 310 in other embodiments.

The biodegradable osteogenic scaffold 330 may be formed as an elongate structure having a proximal end 332 and a distal end 334 disposed opposite one another along the longitudinal axis of the biodegradable osteogenic scaffold 330. In some embodiments, the biodegradable osteogenic scaffold 330 may have a gyroid topology (similar to that depicted in FIG. 2C), with a plurality of interconnected pores. Other porous topologies of the biodegradable osteogenic scaffold 330 may be used in other embodiments. In some embodiments, the biodegradable osteogenic scaffold 330 may have a porosity of 70%. In some embodiments, as shown, the biodegradable osteogenic scaffold 330 may include a scaffold passage 338 extending from the proximal end 332 to the distal end 334 and configured for receiving a collagen sponge carrying rhBMP-2 therein to facilitate bone growth during use of the device 300. However, in some implementations, the scaffold is coated in the BMP, for example, the BMP is poured over the scaffold or the scaffold is dipped into or rolled in a fluid including BMP. In some embodiments, the length of the biodegradable osteogenic scaffold 330 may be less than the length of the biodegradable sleeve 310. In this manner, the biodegradable osteogenic scaffold 330 may be positioned entirely within the biodegradable sleeve 310 during use of the device 300. As discussed above, the biodegradable osteogenic scaffold 330 may be received within the first passage 316 of the biodegradable sleeve 310. In some embodiments, as shown, a portion of the biodegradable osteogenic scaffold 330 may extend into the gap 324 of the biodegradable sleeve 310. In some embodiments, the biodegradable osteogenic scaffold 330 may be formed of a calcium phosphate, such as HAp or 3-TCP. In some embodiments, the biodegradable osteogenic scaffold 330 may be formed of a calcium phosphate-based material or a composite comprising a calcium phosphate and a polymer. Other suitable biocompatible, biodegradable, and osteogenic materials may be used for the biodegradable osteogenic scaffold 330 in other embodiments.

The fixation member 340 may be formed as an elongate structure having a proximal end 342 and a distal end 344 disposed opposite one another along the longitudinal axis of the fixation member 340. According to various embodiments, the fixation member 340 may be a fixation plate, a fixation rod, a locking plate, a locking rod, or other type of suitable structure for fixation of bone structures. In some embodiments, as shown, the fixation member 340 may have a rectangular cross-sectional shape (i.e., taken perpendicular to the longitudinal axis of the fixation member 340). As discussed below, the fixation member 340 may be coupled to the biodegradable sleeve 310 by sliding the biodegradable sleeve 310 onto the fixation member 340 such that the fixation member 340 extends through the second passage 318. As shown, the fixation member 340 may include a plurality of screw holes 346 extending therethrough and spaced apart from one another along the length of the fixation member 340. As discussed below, the screw holes 346 may be used to facilitate connection of the fixation member 340 to the proximal bone structure PBS and the distal bone structure DBS using the screws 360. The fixation member 340 may include a proximal end portion 352, a distal end portion 354, and an intermediate portion 356. As shown, when the fixation member 340 is coupled to the biodegradable sleeve 310, the proximal end portion 352 may extend beyond the proximal end 312 of the biodegradable sleeve 310, the distal end portion 354 may extend beyond the distal end 314 of the biodegradable sleeve 310, and the intermediate portion 356 may be positioned within the second passage 318 of the biodegradable sleeve 310. As shown, the proximal end portion 352 may include a plurality of the screw holes 346 to facilitate connection of the fixation member 340 to the proximal bone structure PBS, and the distal end portion 354 may include a plurality of the screw holes 346 to facilitate connection of the fixation member 340 to the distal bone structure DBS. In some embodiments, the intermediate portion 356 may be devoid of any screw holes 346. In some embodiments, the intermediate portion 356 may include a plurality of the screw holes 346, which may be filled with respective plugs to prevent bone growth therein during use of the device 300. The fixation member 340 may be formed of any suitable biocompatible, non-biodegradable material such that the fixation member 340 may be removed from the patient after the bone defect has healed and the biodegradable sleeve 310 and the biodegradable osteogenic scaffold 330 have degraded. According to various embodiments, the fixation member 340 may be formed of a metal, a polymeric material, a ceramic material, a composite material, or other suitable material for fixation of bone structures.

As noted above, the biodegradable sleeve 310 and the biodegradable osteogenic scaffold 330 may be fabricated as patient-specific components. In some embodiments, CT scans of the patient's limb may be used to fabricate the biodegradable sleeve 310 and the biodegradable osteogenic scaffold 330 for the patient. In some embodiments, a series of two-dimensional DICOM images of the full extent of the limb may be imported (e.g., using In Vesalius) and segmented to create high-resolution digital models of the radius, ulna, manus, and metacarpals of the patient. The images then may be segmented based on Hounsfield attenuation values, and surface representations of the bone may be generated and then imported (e.g., into Fusion 360) where 3D models of the components may be created for 3D printing. In some embodiments, finite element analysis (FEA) may be used to investigate the ideal design parameters of the biodegradable sleeve 310 and the overall device 300, with and without the fixation member 340, to determine optimal geometry of the components and features thereof. Parameters such as wall thickness and mechanical properties such as structural modulus may be simulated to optimize the design of the device 300 prior to fabrication. In some embodiments, the CT scans also may be used to fabricate cutting guides to assure accurate surgical resections to match the geometry of the device 300.

The biodegradable osteogenic scaffold 330 may be 3D printed using existing methods. In some embodiments, a photopolymer comprised of ethylene glycol dimethacrylate (EDGMA) and other polymers may be mixed with hydroxyapatite (HAp) particles into a shear-thinning slurry using a planetary ball mill. Slurries of 41 vol % HAp may be transferred to a syringe and 3D printed on 3D printer, such as a HyRel Engine S R printer with a 0.413 mm nozzle inner diameter, using a combination of viscous extrusion and photopolymer processes. The photopolymerization reaction may be initiated by continuous exposure of the deposited roads to a near-UV light source (405 nm wavelength), causing polymerization and hardening of the continuous phase, layer-by-layer. The sintered biodegradable osteogenic scaffold 330 may be non-cytotoxic. After printing, the biodegradable osteogenic scaffold 330 may undergo a two-step sintering process to eliminate the polymeric content and consolidate the HAp particles. In some embodiments, the printed biodegradable osteogenic scaffold 330 may be heated at 5° C./min up to 500° C. and held for 1 h, and then sintered at a heating rate of 15° C./min, up to 1150° C. and held for 5 h. This process may confer the sintered HAp structures with suitable mechanical properties. In some embodiments, the biodegradable sleeve 310 may be fabricated using a thermal process on a melt extrusion 3D printer (e.g., Prusa i3-MK3, Budapest, Hungary). PCL filament may be extruded by regulated ram pressure through a Flexion head. The extruded PCL may cool on a build plate to fabricate the components, which then may cool to room temperature. Prints may be oriented to maximize part strength and minimize support material.

The device 300 may be used to treat a bone defect, such as a critical-sized bone defect, extending between a proximal bone structure PBS and a distal bone structure DBS of a patient. In some embodiments, as shown in FIGS. 3A and 3B, the bone defect may be an epiphyseal defect. In some embodiments, the bone defect may be a segmental defect. A method of using the device 300 for treating the bone defect generally may include resecting a region of bone between the proximal bone structure PBS and the distal bone structure DBS and encompassing the bone defect, positioning the biodegradable osteogenic scaffold 330 within the biodegradable sleeve 310, coupling the biodegradable sleeve 310 to the fixation member 340, positioning the biodegradable sleeve 310 between the proximal bone structure PBS and the distal bone structure DBS, and attaching the fixation member 340 to each of the proximal bone structure PBS and the distal bone structure DBS.

In some embodiments, the region of bone may be resected using one or more cutting guides fabricated based on CT scans of the patient's limb. For an epiphyseal defect, approximately 40% of the distal radius may be removed, along with a slightly larger section of the ulna to enable insertion of the device 300. In some embodiments, the biodegradable osteogenic scaffold 330 may be treated with one or more bioactive agents to facilitate bone growth or angiogenesis before positioning the biodegradable osteogenic scaffold 330 within the biodegradable sleeve 310. For example, the biodegradable osteogenic scaffold 330 may be treated with rhBMP-2 to facilitate bone growth and/or VEGF to facilitate angiogenesis. In some embodiments, one or more collagen sponges containing the one or more bioactive agents may be inserted within the scaffold passage 338 of the biodegradable osteogenic scaffold 330. In some embodiments, the biodegradable osteogenic scaffold 330 may be positioned within the biodegradable sleeve 310 such that the proximal end 332 of the biodegradable osteogenic scaffold 330 is offset from the proximal end 312 of the biodegradable sleeve 310, and a distal portion of the proximal bone structure PBS may be inserted into the first passage 316 of the biodegradable sleeve 310 and in contact with the proximal end 332 of the biodegradable osteogenic scaffold 330. For an epiphyseal defect, a distal portion of the remaining host radius may be inserted into the first passage 316 of the biodegradable sleeve 310 and in contact with the proximal end 332 of the biodegradable osteogenic scaffold 330. In some embodiments, the biodegradable osteogenic scaffold 330 may be positioned within the biodegradable sleeve 310 such that the distal end 334 of the biodegradable osteogenic scaffold 330 is flush with the distal end 314 of the biodegradable sleeve 310, and a proximal portion of the distal bone structure DBS may be positioned in close contact with the distal end 334 of the biodegradable osteogenic scaffold 330 and the distal end 314 of the biodegradable sleeve 310. For an epiphyseal defect, the proximal end of the carpus may be positioned in close contact with the distal end 334 of the biodegradable osteogenic scaffold 330 and the distal end 314 of the biodegradable sleeve 310. In some embodiments, the biodegradable osteogenic scaffold 330 may be positioned within the biodegradable sleeve 310 such that the distal end 334 of the biodegradable osteogenic scaffold 330 is offset from the distal end 314 of the biodegradable sleeve 310, and a proximal portion of the distal bone structure DBS may be inserted into the first passage 316 of the biodegradable sleeve 310 and in contact with the distal end 334 of the biodegradable osteogenic scaffold 330.

In some embodiments, the biodegradable sleeve 310 may be coupled to the fixation member 340 before the biodegradable sleeve 310 is positioned between the proximal bone structure PBS and the distal bone structure DBS. In some embodiments, the biodegradable sleeve 310 may be coupled to the fixation member 340 by sliding the biodegradable sleeve 310 over the fixation member 340 such that the intermediate portion 356 of the fixation member 340 is positioned within the second passage 318, the proximal end portion 352 of the fixation member 340 extends beyond the proximal end 312 of the biodegradable sleeve 310, and the distal end portion 354 of the fixation member 340 extends beyond the distal end 314 of the biodegradable sleeve 310. In some embodiments, the proximal end portion 352 of the fixation member 340 may be attached to the proximal bone structure PBS using a first plurality of the screws 360, and the distal end portion 354 of the fixation member 340 may be attached to the distal bone structure DBS using a second plurality of the screws 360. For an epiphyseal defect, the proximal end portion 352 of the fixation member 340 may be attached to the remaining host radius, and the distal end portion 354 of the fixation member 340 may be attached to the third metacarpal. In some embodiments, the biodegradable sleeve 310 may be coupled to the fixation member 340 and positioned between the proximal bone structure PBS and the distal bone structure DBS after the fixation member 340 is attached to the proximal bone structure PBS but before the fixation member 340 is attached to the distal bone structure DBS. In other words, the proximal end portion 352 of the fixation member 340 may be attached to the proximal bone structure PBS, the biodegradable sleeve 310 then may be slid onto the fixation member 340 from the proximal end 342 toward the distal end 344 and positioned between the proximal bone structure PBS and the distal bone structure DBS, and the distal end portion 354 of the fixation member 340 then may be attached to the distal bone structure DBS.

Noncombat musculoskeletal injuries account for nearly 60% of active component (AC) soldiers' limited duty days and 65% of AC soldiers who cannot deploy for medical reasons. Most bone trauma received by war-fighters are to the long bones caused by numerous common non-combat activities like jumping off vehicles, parachuting, repetitive weight-bearing activities, and motor vehicle-related injuries. Many of these injuries are “critical” defects where the damage to bone that is so large that it will never heal. Poor healing of critical bone defects remains one of the biggest challenges in human orthopedic medicine, affecting more than 1.5 million Americans per year and often leading to infections and other clinical complications, reoperations, poor functional outcomes, huge costs and ultimately, all too often, limb loss. Critical defects become ingrown with soft, bone-like tissue that can never support normal bone function. Critical defects are extremely expensive in terms of military cost, unit readiness, and our Warfighter's general positive mental attitude. There is a need to address this challenge in the DoD and in human medicine overall.

These injuries take a long time to achieve functional rehabilitation, which reduces unit readiness. Metal fixation, a gold-standard treatment, supports the soldier's weight while attempting to remediate the bone defect with bone grafts or fillers. Numerous studies have demonstrated the efficacy of locking plates in restoring quasi-normal limb function, but the bone never heals correctly, limiting any chance of return to active duty. This technique requires that metal remain in the patient for lifetime which is a common source of infection risk and significant discomfort which are both common causes of delayed return to duty. A remedy that would accelerate and improve the quality of bone regrowth would be beneficial, especially if such a remedy might someday reduce or eliminate all metal in the soldier's body.

A viable solution to healing critical defects requires rapid ossification, vascular development, and mechanical integrity to support loads while the bone heals. Healthy bone growth needs high levels of nutrient and waste exchange, especially in large bone defects, which require high levels of perfusion. Tissue engineered solutions have emerged that deploy biodegradable, bioactive (osteoinduction, osteoconduction, and osseointegration) scaffolds with high porosity to enable nutrient and waste exchange and angiogenesis. Tri-calcium-phosphate (TCP) scaffolds are widely used for bone regeneration due to their high levels of bioactivity, tunable degradation rates and promising drug delivery capabilities. These materials are bioreplaceable by new, native bone. They release calcium during degradation, which supports bone formation, resulting in excellent osteoconductivity. However, despite excellent bone regeneration properties, success of these scaffolds, especially in large defects, is hampered by inadequate mechanical properties such as strength and toughness, which are required for load bearing. This limitation is amplified when scaffolds are designed with high-porosity to accommodate adequate nutrient and waste exchange in large bone defects. Efforts to strengthen TCP scaffolds with polymers like polycaprolactone (PCL) have yielded stronger scaffolds, but with reduced osteogenic properties and long degradation times which prevent complete natural remodeling, limits new bone formation, and delays restoration of normal function.

In addition to material improvements to support ossification, physical characteristics like scaffold topology also play an important role. Topologies such as gyroid have emerged in recent decades as promising open-cell architectures for bone TE scaffolds due to their high surface area to volume ratio and high relative strength, anisotropic elastic properties, and continuous, interconnected porosity which enable higher permeability and perfusion than other porous morphologies. These properties enable high porosity, ideal pore size, and high permeability which are important factors for cell migration and proliferation, and improved stiffness to support higher loads. Studies have demonstrated advantageous in vitro and in vivo bioactivity and mechanical performance of gyroid TCP scaffolds over common rectilinear scaffolds.

Many researchers have approached the critical defect healing problem with tissue engineered approaches. Successful healing involves a careful balance between scaffold degradation and tissue remodeling and maturation. Some current devices use composite rectilinear PCL/TCP scaffolds, with PCL the primary material (% volume), to provide additional strength for load bearing while the tissue was remodeled and matured. The brittle TCP can require substantial strength because it is sometimes press-fit into the defect where it remains unprotected. PCL may take approximately 2 years to degrade, thereby rendering improbable remodeling of the majority of the scaffold because it is filled with PCL, which is much weaker than cortical bone. While some progress has created stronger scaffolds, these devices compromise the rate at which ossification can occur and newly formed bone can mature. While some of these devices have successfully bridged a critical defect site, none have succeeded in improving bending stiffness compared to their controls, even after 16 months, which means more work is needed to improve the activity and load bearing of an active-duty warfighter.

The devices and methods disclosed herein provide a surgical method and technology that improve high-energy extremity trauma care and can facilitate the ability for warfighters to return to duty/work in less the one year. The endoprosthetic devices, systems, and methods herein described augment the standard locking compression plate with a novel endoprosthetic “sleeve” device that contains and protects a highly osteogenic, pure 3-TCP scaffold. The LCP bears the load, as in the current gold-standard remediation.

Other devices have not used an endoprosthetic “sleeve” device to protect and immobilize a fragile but highly osteogenic scaffold to maximize the rate of ossification in the defect site under human-scale loads. Rigid stabilization of the scaffold by the sleeve at the host-bone interfaces increases opportunity for primary ossification to occur in these transition zones. Concurrently, transverse micromotion induced by the sleeve exerts strain on the body of the scaffold, which is shown to accelerate callus formation, a key step in the bone healing process. Micro-motion has been shown to increase mineralization, decrease the formation of unwanted cartilage, and influence early bone regeneration at the bone-implant interface in appropriate magnitudes. By moving all structural support outside the bone/scaffold construct, the systems, devices, and methods herein restore normal intramedullary perfusion and normal centrifugal flow, which accelerates vascularization and enables higher rates of bioactivity and ossification. Using gyroid scaffolds instead of rectilinear topology is shown to improve bioactivity and mechanical performance. Moreover, enhancing periosteal engagement accelerates healing by guiding periosteal stem cells (PSCs) into the scaffold instead of dissemination into the surrounding interstitia. Ultimately, a homogeneous distribution of mature bone aids in complete healing and full recovery. Because there is little or no PCL in the scaffolds of this disclosure, bone tissue comprises approximately 100% of total defect volume sooner, thereby increasing mechanical strength faster. Meanwhile, the large portals in the sleeves disclosed herein are designed to avoid interference with normal cross-cortical flow and inter- and extra-medullary engagement in the healing process.

The approaches disclosed herein could relieve a warfighter or other injured person from the pain, discomfort, and risk of a lifetime with metal in the body. All or most materials proposed in this disclosure are fully biodegradable, and the sleeve is designed to enable the plate to be removed from the sleeve when the endogenous bone has matured.

The endoprosthetic system of this disclosure has been designed and optimized to (1) prohibit scaffold migration in the scaffold/host bone transition zones; (2) allow maximum material flow and subsequent nutrient exchange between the intra- and periosseous regions; (3) optimize plated-construct torsional rigidity; (4) temporarily constrain scaffold positioning until adequate callous formation and ossification occur, while simultaneously (5) induce micro-motion into the scaffold body. Patient-specific CT scans are utilized to generate appropriate sleeve dimensions (i.e., internal shape and dimensions). The endoprosthetic sleeve device extends between and simultaneously attaches to a small portion of both the proximal and distal aspects of the host bone and the LCP.

Windows placed in the sleeve walls alongside the defect region are optimized to enable stem cell cross-migration and nutrient exchange within the defect site while simultaneously improving torsional stiffness of the plated construct via finite element analysis (FEA) simulations. To that end, simulations implementing this sleeve design on a plated ovine tibia construct (4.5 mm broad 11-hole LCP) revealed a 9.5% increase in torsional stiffness as compared to separate cadaveric biomechanical testing results, suggesting the endoprosthetic device/plated construct can more closely approximate the mechanical stability of an in-tact limb.

Micro-motion has been shown to increase mineralization, decrease the formation of unwanted cartilage, and influence early bone regeneration at the bone-implant interface in appropriate magnitudes. For those reasons, the body of the sleeve is designed to induce transverse micromotion by compressing the body of the scaffold while simultaneously restricting excessive scaffold motion. While the level of strain is impacted by patient-specific factors (i.e., bone size and patient weight/loading), preliminary FEA using ovine tibia size and loads revealed approximately 1.0% of transverse strain will be applied to the scaffold mid-body during ambulation. Interfragmentary strain less than 2% have been shown to exert a positive influence on ossification.

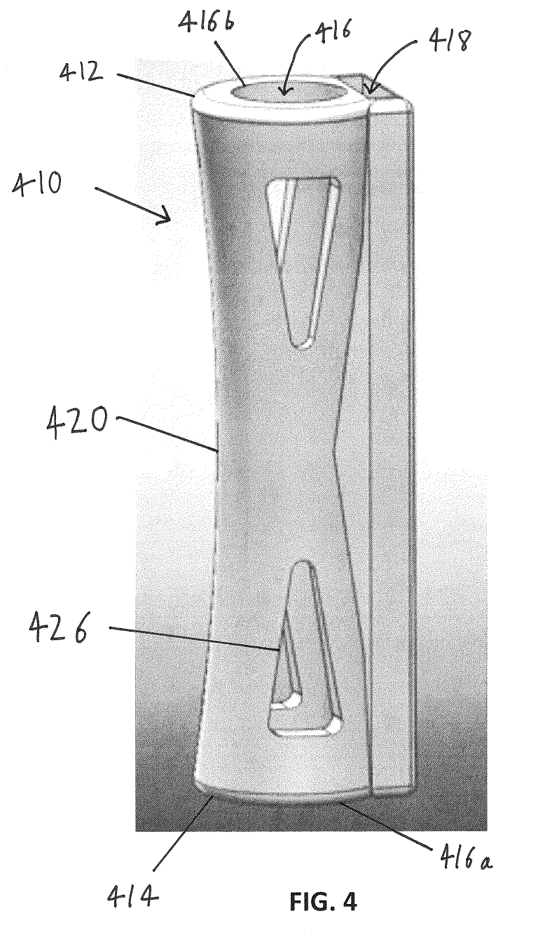

The devices shown in FIGS. 4-8 include biodegradable sleeves that have various geometries that are predetermined to induce strain on a portion of a biodegradable scaffold when a force is applied to the biodegradable sleeve. Such geometries can create areas of relatively higher and lower rigidity to allow one or more portions of the biodegradable sleeve to flex or move. Examples of geometrical features of biodegradable sleeves that can induce strain on a biodegradable scaffold can include differences in wall thickness of the biodegradable sleeve, ribbed portions of the outer or inner surface of the biodegradable sleeve, a concavity of the wall of the biodegradable sleeve, differences in material included in portions of the wall of the biodegradable sleeve, reinforced portions of the wall of the biodegradable sleeve, and/or any other feature of the wall or another portion of the biodegradable sleeve that causes the biodegradable sleeve to move toward or transfer force to the biodegradable scaffold when force is applied to the biodegradable sleeve. The strain induced by the biodegradable sleeve on the biodegradable scaffold causes micromotion in the biodegradable scaffold, which can increase mineralization, decrease the formation of unwanted cartilage, and influence early bone regeneration at the bone-implant interface.

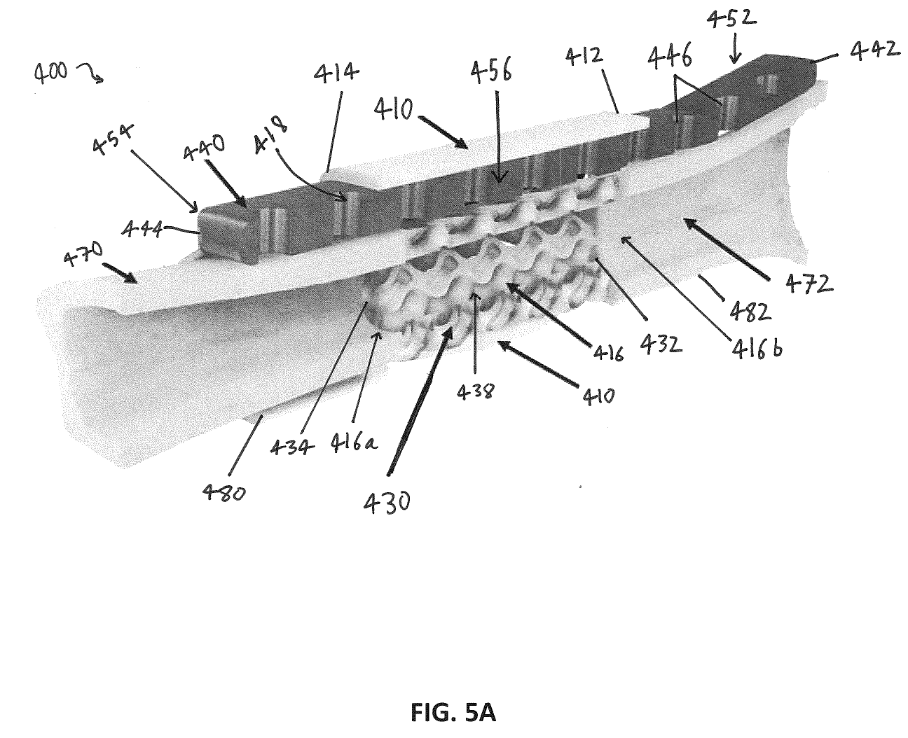

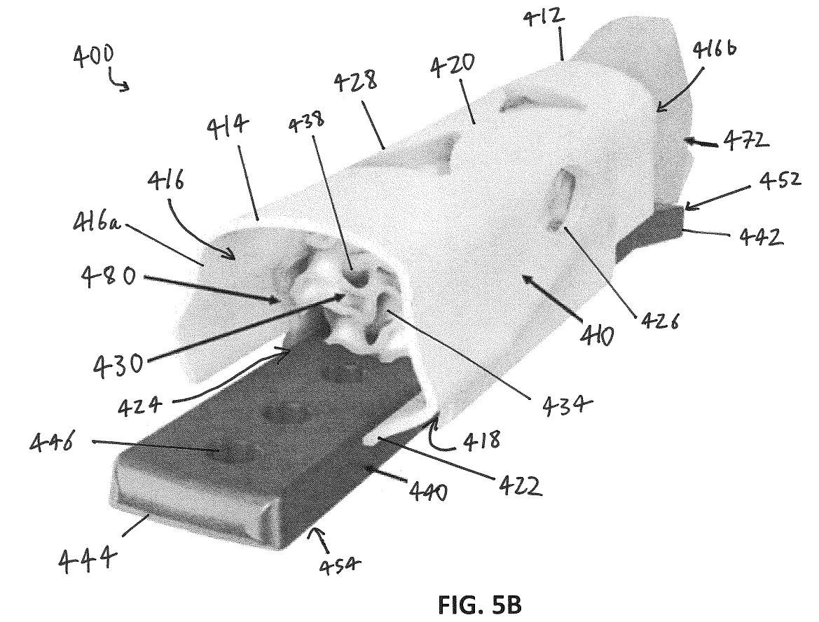

FIGS. 4, 5A, and 5B show another device for treating a bone defect extending between a first bone structure and a second bone structure of a patient. The device includes aspects according to various implementations. The device shown in FIGS. 4, 5A, and 5B is similar to those shown in FIGS. 1-3, and it is contemplated that features of the devices shown in FIGS. 1-3 can be incorporated in addition to, in combination with, and/or in replacement of one or more features of the device shown in FIGS. 4, 5A, and 5B to form various other implementations according to the disclosure herein.

The device 400 shown in FIGS. 4, 5A, and 5B includes a biodegradable osteogenic scaffold 430, a biodegradable sleeve 410, and a fixation member 440.

The biodegradable sleeve 410 is configured to be positioned between a first bone structure 470 and a second bone structure 472. The biodegradable sleeve 410 has a first end 412 and a second end 414 opposite and spaced apart from the first end 412. The first end 412 of the biodegradable sleeve 410 defines a first passage 416 and a second passage 418 each extending to the second end 414 of the biodegradable sleeve 410. The first passage 416 is configured for receiving the biodegradable osteogenic scaffold 430 and for receiving an end portion of the first bone structure 470 and an end portion of the second bone 472 structure therein. The second passage 418 is configured for receiving a portion of the fixation member 440 therein.

The first end 416a of the first passage 416 of the biodegradable sleeve 410 further defines a first periosteum slot 480, and the second end 416b of the first passage 416 of the biodegradable sleeve 410 defines a second periosteum slot 482. The first periosteum slot 480 is configured for insertion of an end portion of periosteum associated with the first bone structure 470, and the second periosteum slot 482 is configured for insertion of an end portion of periosteum associated with the second bone structure 472. The first periosteum slot 480 and the second periosteum slot 482 are located at a portion of the biodegradable sleeve 410 where the periosteum can be inserted to guide movement of periosteal exudate (containing such materials as periosteal stems cells and proteins associated with bone healing) into the biodegradable osteogenic scaffold 430 to accelerate bone healing.

The outer surface of the biodegradable sleeve 410 defines a concavity 420 extending radially inwardly. The outer surface of the biodegradable sleeve 410 defines a concavity 420 extending radially inwardly toward the first passage 416. The concavity 420 creates portions of varying wall thickness, as measured from an outer surface of the sleeve to an inner surface of the sleeve, along the length of the sleeve 410. Because the two or more portions of the wall of the sleeve 410 have different thickness, each of the two or more portions of the wall have different rigidity. The geometry of the concavity 420 of the biodegradable sleeve 410 is configured such that, when a force is applied to the biodegradable sleeve 420, the different rigidity portions of the wall cause the sleeve 410 to contact and induce strain on a portion the biodegradable scaffold 430. The protrusion 420 shown in FIGS. 4, 5A, and 5B is formed from a convex portion of the wall of the biodegradable sleeve 410 as viewed from inside the first passage 416 (i.e., concave portion of the wall of the biodegradable sleeve 410 as viewed from a position external to the biodegradable sleeve 410).

Although the biodegradable sleeve 410 shown in FIGS. 4-5B defines a concavity 420 that creates two or more portions of the wall of the sleeve 410 having different rigidity, in some implementations, the biodegradable sleeve can include one or more portions of the wall that include a material that is not included in one or more other portions of the wall of the sleeve. In these implementations, one portion of the wall of the sleeve could include a more rigid material or combination of materials than another portion of the wall of the sleeve. The different portions of rigidity of the wall can be configured in a geometry that induces strain on a portion of the biodegradable scaffold when a force is applied to the biodegradable sleeve.

Furthermore, in some implementations, the biodegradable sleeve can include any feature that creates a portion of the sleeve to be more rigid than another portion of the sleeve such that it induces strain on a portion of the biodegradable scaffold when a force is applied to the biodegradable sleeve. In some implementations, the biodegradable sleeve can include any geometry that induces strain on a portion of the biodegradable scaffold when a force is applied to the biodegradable sleeve.

For example, in some implementations, such as the implementations shown in FIGS. 7 and 8, the biodegradable sleeve 710, 810 can include one or more ribs 717, 817. The ribs 717, 817 can be similar to the support ribs 422, or the ribs 717, 817 can extend from the outer surface of the wall of the sleeve 710, 810. Similar to the concavity 420 of the biodegradable sleeve 410 shown in FIGS. 4-5B, the geometry of the one or more ribs 717, 817 can create portions of the wall of the biodegradable sleeve 710, 810 that have different wall thicknesses. This geometry can be designed to induce strain on a portion of the biodegradable scaffold 730, 830 when a force is applied to the biodegradable sleeve 710, 810.

In FIG. 7, the one or more ribs 717 include a first rib 717 adjacent the first end 712 of the biodegradable sleeve 710 and a second rib 717 adjacent the second end 414 of the biodegradable sleeve 710. The biodegradable sleeve 710 further includes ribs 717 extending diagonally across the biodegradable sleeve 710 relative to the longitudinal axis of the biodegradable sleeve 710 and a rib 717 extending across the center of the biodegradable sleeve 710 perpendicular to the longitudinal axis of the biodegradable sleeve 710. The diagonally extending ribs 717 and the center rib 717 intersect in the middle of the sleeve.

When the bone (i.e., the mandible) is loaded by a force, the force is transferred to the fasteners that couple the biodegradable sleeve 710 to the bone. The force is then transferred from the fasteners to the biodegradable sleeve 710. The geometry of the ribs 717 cause the biodegradable sleeve 710 to deflect in a predetermined way such that the biodegradable sleeve 710 induces strain on a portion of the biodegradable scaffold 730 to cause micromotion.

Similarly, the biodegradable sleeve 810 includes ribs adjacent the first end 812 and the second rib 814 of the biodegradable sleeve 810. The biodegradable sleeve 810 also includes a rib 817 extending across the center of the biodegradable sleeve 810 perpendicular to the longitudinal axis of the biodegradable sleeve 810.

Like the biodegradable sleeve 710 of FIG. 7, when the bone (i.e., the mandible) is loaded by a force, the force is also transferred to the fasteners that couple the biodegradable sleeve 810 to the bone. The force is then transferred from the fasteners to the biodegradable sleeve 810. The geometry of the ribs 817 cause the biodegradable sleeve 810 to deflect in a predetermined way such that the biodegradable sleeve 810 induces strain on a portion of the biodegradable scaffold 830 to cause micromotion.

For the biodegradable sleeve 810 shown in FIGS. 4-6, the curved surface of the concavity 420 extends along a centerline that extends from the first end 412 of the biodegradable sleeve 410 to the second end 414 of the biodegradable sleeve 410. However, in some implementations, the concavity 420 can be any shape or any length capable of causing the wall of the biodegradable sleeve 410 to flex when force is applied such that a portion of the inner surface of the biodegradable sleeve 410 contacts at least a portion of the biodegradable osteogenic scaffold 430 when disposed within the first passage 416. In some implementations, the concavity is a portion of the wall of the biodegradable sleeve that has a greater thickness such that the wall extends further into the first passage than other portions of the wall of the biodegradable sleeve.

Rigid stabilization of the biodegradable osteogenic scaffold 430 by the biodegradable sleeve 410 at the bone-implant interfaces can increase the opportunity for primary ossification to occur in these transition zones. Furthermore, transverse micromotion induced by the biodegradable sleeve 410 can exert a strain on the body of the biodegradable osteogenic scaffold 430. The concavity 420 in the biodegradable sleeve 410 aids in exerting the strain on the scaffold 430, producing micromotion. Micromotion at this bone-implant interface is shown to accelerate callus formation, a key step in the bone healing process. Micromotion has also been shown to increase mineralization, decrease the formation of unwanted cartilage, and influence early bone regeneration at the bone-implant interface in appropriate magnitudes.

The biodegradable sleeve 410 further includes support ribs 422 disposed between the first passage 416 and the second passage 418 and extending from the first end 412 to the second end 414 of the biodegradable sleeve 410. Because the support ribs 422 are spaced apart from each other, the first passage 416 is in communication with the second passage 418 through a gap 424 defined between the support ribs 422. When the biodegradable osteogenic scaffold 430 is positioned within the biodegradable sleeve 410, a portion of the biodegradable osteogenic scaffold 430 extends into the gap 424 between the support ribs 422.

In some implementations, the support ribs 422 facilitate placement and engagement with the fixation members 440. In some implementations, the support ribs 422 may facilitate connection of the biodegradable sleeve 410 to the biodegradable cuffs (shown in FIG. 2), facilitating load sharing therebetween. In some implementations, the support ribs can induce micromotion by transferring force applied to the biodegradable sleeve to the biodegradable osteogenic scaffold 430. In some embodiments, each of the biodegradable cuffs may include a plurality of protrusions extending radially inward and configured for mating with the support ribs of the biodegradable sleeve. The mating engagement between the protrusions and the support ribs may facilitate alignment of the screw holes of the biodegradable cuff with the screw holes of the biodegradable sleeve and the screw holes of the fixation member.

The biodegradable sleeve 410 further comprises a plurality of apertures 426 disposed along one or more sides of the biodegradable sleeve 410 and extending from an outer surface of the biodegradable sleeve 410 to the first passage 416. The apertures 426 are configured for controlling transcortical perfusion.

The biodegradable sleeve 410 further includes a plurality of openings 428 disposed along a side of the biodegradable sleeve 410 and extending from an outer surface of the biodegradable sleeve 410 to the second passage 418. The openings 428 expose respective portions of the fixation member 440 when the fixation member 440 is coupled to the biodegradable sleeve 410. The openings 428 are configured for receiving collagen sponges carrying an antibiotic therein.

Although the biodegradable sleeve 410 shown in FIGS. 4, 5A, and 5B includes support ribs 422, apertures 426, and openings 428, in some implementations, the biodegradable sleeve includes any other geometric features, in addition to, instead of, or in combination with the support ribs, the apertures, and/or the openings, that can affect the performance of the device. In some implementations, the biodegradable sleeve includes solid walls with no support ribs, apertures, and/or openings.

The biodegradable sleeve 410 shown in FIGS. 4, 5A, and 5B is formed of polycaprolactone, but in some implementations, the biodegradable sleeve is formed of a composite including a calcium phosphate and polycaprolactone.

The biodegradable osteogenic scaffold 430 has a first end 432 and a second end 434 opposite and spaced apart from the first end 432 of the biodegradable osteogenic scaffold 430. The first end 432 of the biodegradable osteogenic scaffold 430 defines a scaffold passage 438 extending to the second end 434 of the biodegradable osteogenic scaffold 430. The central opening of the scaffold passage 438 of the biodegradable osteogenic scaffold 430 shown in FIGS. 4, 5A, and 5B is configured for receiving a collagen sponge carrying bioactive agents. Although the collagen sponge shown in FIGS. 4, 5A, and 5B includes BMP, in some implementations, the scaffold passage could be used for deploying VEGF. In some implementations, the central opening utilizes clotting to deploy a bioactive agent such as BMP without a collagen sponge. In some implementations, the bioactive agents are rhBMP-2, VEGF, or any known type of bioactive agent.

The biodegradable osteogenic scaffold 430 shown in FIGS. 4, 5A, and 5B is formed of a calcium phosphate-based material, but in some implementations, the biodegradable osteogenic scaffold is formed of or a composite including a calcium phosphate and a polymer.

Although the biodegradable scaffold 430 shown in FIGS. 4, 5A, and 5B is a biodegradable osteogenic scaffold 430, in some implementations, the biodegradable scaffold is useful with bone-void-filling putties as a way of helping recreate long bone “shape” in procedures such as critical defect repair surgeries. In such procedures, the sleeve acts as a mold for the surgeon to fill with bone-void-filling putty. The bone-void-filling putty ensures the biodegradable scaffold stays in place and forms bone in the correct shape.

The fixation member 440 is configured for coupling to the biodegradable sleeve 410 and coupling to each of the first bone structure 470 and the second bone structure 472. The fixation member 440 includes a first portion 452, a second portion 454, and an intermediate portion 456. The first portion 452 extends beyond the first end 412 of the biodegradable sleeve 410 when the fixation member 440 is coupled to the biodegradable sleeve 410. The second portion 454 extends beyond the second end 414 of the biodegradable sleeve 410 when the fixation member 440 is coupled to the biodegradable sleeve 410. The intermediate portion 456 is positioned within the second passage 418 when the fixation member 440 is coupled to the biodegradable sleeve 410.

Although the device 400 shown in FIGS. 4, 5A, and 5B includes a fixation member 440, in some implementations such as less load-bearing cases, the device does not include a fixation device and can be coupled to the first bone structure and the second bone structure in any other way known in the art (e.g., bone cement). In such implementations, the biodegradable sleeve may not include a second passage.

In use, computed tomography scans are first obtained of the patient. A biodegradable osteogenic scaffold 430 and a biodegradable sleeve 410, as described above, can then be fabricated based at least in part on the computed tomography scans. Also, a cutting guide can be fabricated based at least in part on the computed tomography scans.

Next, using the cutting guide, a region of bone between the first bone structure 470 and the second bone structure 472 can be resected and the bone defect encompassed. However, in some implementations, the bone defect does not require resection of the bone, and this step can be skipped.

The biodegradable osteogenic scaffold 430 is then positioned within the first passage 416 of a biodegradable sleeve 410. As discussed above, the biodegradable osteogenic scaffold 430 can be treated with one or more bioactive agents to facilitate osteoinduction, osteoconduction, osteointegration, vascularization, or angiogenesis.

The biodegradable sleeve 410 is then coupled to the fixation member 440 by inserting the fixation member 440 into the second passage 418 of the biodegradable sleeve 410. The fixation member 440 is located in the second passage 418 such that the first portion 452 of the fixation member 440 extends from the first end 412 of the biodegradable sleeve 410 and the second portion 454 of the fixation member 440 extends from the second end 414 of the biodegradable sleeve 410. The fixation member 440 can be treated with an antibiotic to inhibit infection.

The biodegradable sleeve 410 is positioned between the first bone structure 470 and the second bone structure 472. An end portion of the first bone structure 470 is inserted into the first end 412 of the biodegradable sleeve 410 such that the end portion of the first bone structure 470 contacts the biodegradable osteogenic scaffold 430. Then, an end portion of the second bone structure 472 is inserted into the second end 414 of the biodegradable sleeve 410 such that the end portion of the second bone structure 472 contacts the biodegradable osteogenic scaffold 430. The contact between the ends of the biodegradable osteogenic scaffold 430 contacting the bone structure 470, 472 allows the biodegradable sleeve 410 to immobilize the interface between biodegradable osteogenic scaffold 430 and the bone structure 470, 472. As with the fixation member 440, the biodegradable sleeve 410 can be treated with an antibiotic to inhibit infection.

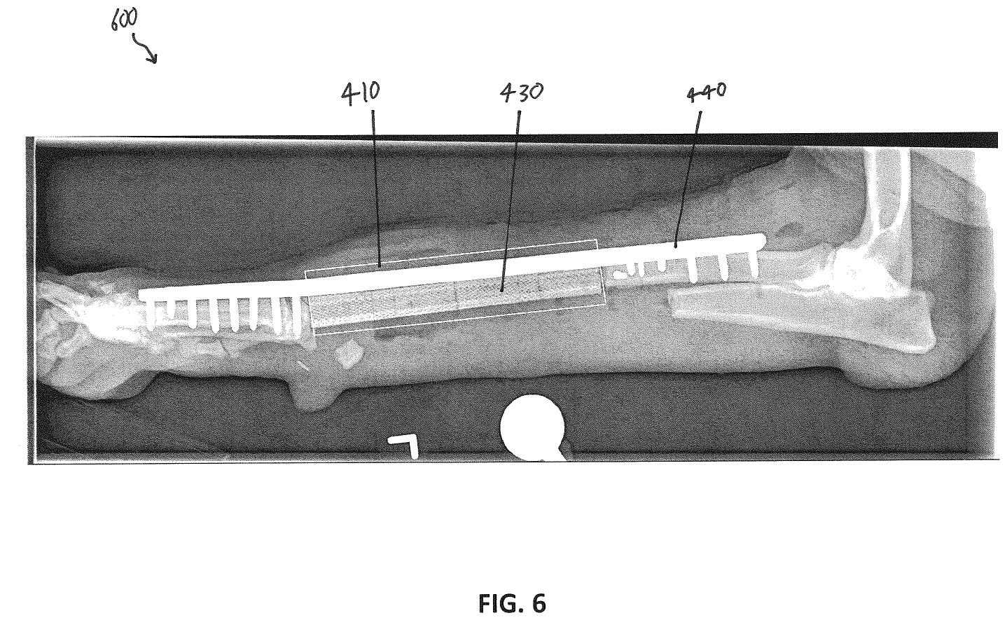

The first portion 452 of the fixation member 440 is then coupled to the first bone structure 470, and the second portion 454 of the fixation member 440 is coupled to the second bone structure 472. To attach the fixation member 440 to the first bone structure 470 and the second bone structure 472, a plurality of screws (as shown in FIG. 6) can be fastened through the first portion 452 and the second portion 454 of the fixation member 440 into the first bone structure 470 and the second bone structure 472, respectively, for example, at screw holes 446. However, in some implementations, the fixation member is not attached to the first bone structure and the second bone structure using screws, and instead, the fixation member is attached to the first bone structure and the second bone structure using bone cement or any other coupling means known in the art. In some implementations, no fixation member is used.

As discussed above, the geometry of concavity creates portions of different wall thicknesses along the biodegradable sleeve 410. The concavity 420 of the wall of the biodegradable sleeve 410 causes the biodegradable sleeve 410 to flex during loading of the first bone structure 470 or second bone structure 472 such that the inner surface of the wall of the biodegradable sleeve 410 exerts strain on the biodegradable osteogenic scaffold 430. The strain exerted due to the concavity 420 induces micromotion in the biodegradable osteogenic scaffold 430, which accelerates callus formation. However, because the first bone structure 470 and the second bone structure 472 are coupled to the first portion 452 and the second portion 454 of the fixation member 440, respectively, the first bone structure 470 and second bone structure 472 remain static relative to the biodegradable sleeve 410 during loading of the first bone structure 470 or second bone structure 472.

Once the bone defect has healed and the biodegradable osteogenic scaffold 430 and the biodegradable sleeve 410 have degraded, the fixation member 440 can be detached from the first bone structure 470 and the second bone structure 472. The fixation member 440 can then be removed from the patient. In cases where no fixation member is used, removal of any components may not be necessary. If plug devices are inserted into unused screw holes in the fixation member when being coupled to the first bone structure and the second bone structure, the plug devices can facilitate the removal of the fixation member after the bone defect has healed and the biodegradable osteogenic scaffold and the biodegradable sleeve have degraded.

### Examples

A workflow was developed wherein high precision computed tomography (CT) scans were taken of patients for 3D printing of custom endoprostheses, scaffolds and surgical cutting guides. A series of two-dimensional DICOM images of the metatarsi were segmented using AMIRA® visualization software (VSG, Burlington, MA) to create 3D digital surfaces. The surfaces were then imported into Autodesk® Fusion 360® (Autodesk, Sausalito, CA) computer-aided design (CAD) software and 3D digital models of patient morphology were created for 3D printing. Any necessary repairs to the models were done in Fusion 360 to produce manifold models ready for 3D printing that fit the patient defect site precisely. The imagery was also used to create surgical cutting guides that register to specific patient bone morphology to assure precise excision of the host bone to produce a tight fit between the scaffold and the proximal and distal host bone, which is expected to enhance ossification.

A method of viscous extrusion 3D printing was adapted that is referred to herein as photocasting, which combines robocasting with layer-wise curing of a photopolymer resin consisting of various calcium phosphate-based ceramic powders. This method enables fabrication of high-precision scaffolds in very complex topologies such as the gyroid shown in FIGS. 5A and 5B which was used in canine pilot clinical trials. Printed objects were sintered to remove all polymeric content and to densify. The resulting objects exhibited the compressive strength in the low range of cancellous bone.

It should be noted that, although the present example of fabrication uses viscous extrusion 3D printing, the devices and systems disclosed herein are not limited to melt extrusion. For example, in some implementations, one or more parts of the devices and systems disclosed herein can be formed using a powder-bed fusion 3D printer, by sintering the PCL and PCL/TCP composite, or by any other method known in the art.

Pure calcium phosphate-based scaffolds are highly osteogenic but lack sufficient mechanical properties to support any appreciable load. The methods disclosed herein afford excellent, precise control of shape factors like pore size, volume fraction, percent porosity, and pore shape, which have a direct effect on the mechanical properties of scaffolds. The scaffolds disclosed herein have approximately 70% effective porosity with approximately 300 μm to 400 μm interconnected pores, which are considered ideal to effectively support osteoinduction. The scaffolds disclosed herein precisely fit the defect site. Well-fitting scaffolds increase the tendency toward primary ossification as a result of interfragmentary strain less than 2%. Moreover, a close tolerance between the implant and host bone enables loads to be transmitted more evenly throughout the construct.

Stiffness of the endoprosthetic system installed in whole canine cadaveric forelimbs was measured to (1) characterize strain of the sleeve and scaffold in the defect relative to the host bone, (2) identify any (unexpected) adverse effects on the normal performance of the locking compression plate (LCP) as installed in a standard limb-sparing procedure, and (3) to gather mechanical performance metrics. To discern between effects, strains were measured on three configurations: (1) LCP only, (2) LCP and scaffold (press fit into defect) and (3) LCP, scaffold, and sleeve (the full endoprosthetic system).

Six canine cadaver limbs were selected from a pool of twelve cadavers (greater than 140 lb dogs) at the surgeon's discretion and complete device constructs (locking plate, sleeve, scaffold) were installed using a common limb-spare surgical procedure. Scaffolds were 8 cm in length and shape-matched to a typical canine radius. Each leg was dissected so that the humerus could be potted and fit into a custom mount for an MTS 370 Load Frame equipped with an axial-torsional Load Transducer (MTS System).

The limbs were placed onto the load frame with the paws at an approximately 60° from horizontal and the elbows extended to approximately 125° to best simulate the mechanical load experienced by the leg of a trotting dog.

Loads of 300, 700 and 1200N were applied to represent low, medium, and high live loading conditions. Twenty pre-load cycles of 100N compression were applied before each test, followed by 3 second holds for each test. High-speed video was taken during each test to enable quantification of relative displacements.

The ultimate walking and trotting curves were isolated and fit to linear trendlines from which slopes were recorded to determine the full construct stiffness using a prior method. From the motion-capture video, it was confirmed that less than 1 mm of displacement in all cases and test conditions. For this scaffold size, this equates to less than 2% strain at the host bone/sleeve interface, which can have a positive influence on primary ossification. No visible damage was inflicted on the scaffolds during testing and the scaffold remained in intimate contact with proximal and distal host bone, indicating that the sleeve performed well immobilizing and protecting the scaffolds.

Micro-computed tomography (CT) imaging was performed using a Scanco 80 (Scanco Medical A G, Bruttisellen, Switzerland) to assess internal structure and measure pore size and interconnected porosity of our scaffolds. The resolution for the scan was 37 μm. Three 2×1×1 cm 70% porous gyroid scaffolds designed for permeability testing and four 70% porous scaffolds matching the shape of the radius used in the canine clinical trial were scanned. Values for total volume, material volume, mean spacing between struts, and mean strut thickness were returned by the device. The effective porosity of the scaffolds was approximately 70% with approximately 300 μm to 400 μm interconnected pores. Both of these metrics are considered ideal to support osteoinduction. Permeability was measured to quantify the scaffold's ability to conduct fluid. It provides a comprehensive look into many correlated properties relevant to tissue engineering, such porosity, pore shape, pore size and interconnectivity. This is a consideration when evaluating the likelihood that a scaffold might encourage bone regeneration to ensure that the scaffolds enable sufficient flow of interstitial fluid and nutrients for revascularization and bone regrowth. The permeability of the gyroid scaffolds disclosed herein were found to be comparable to the physiological permeability of human trabecular bone (0.4−11×10−9 m2).

Tests and studies of this disclosures were performed to validate the endoprosthetic system in a pilot limb-sparing study using large client-owned dogs (approximately 160 pounds) with super-critical radial defects resulting from distal radial osteosarcoma. Osteotomies removed up to 40% of the patient's distal radius to the manus. Patient C T imagery was segmented using the method described above (Patient-Specific Device Modeling) to provide accurate 3D geometry, from which CAD models were generated to fabricate patient-specific endoprostheses, scaffolds, and cutting guide using the methods described above. A workflow was developed and standardized to ensure delivery of final sterilized devices within four days from receipt of patient CT imagery. LCP fixation was used. A surgical procedure was also developed and tested on canine cadaver limbs to ensure easy installation of the system without substantial change to the standard LCP implantation procedure.

The canine pilot study is a proof of concept of the disclosed endoprosthetic system. Because the animals were presented with osteosarcoma, they received anti-cancer therapy, which is expected to inhibit normal ossification processes. Rather, the objectives of the study described herein were to (1) validate that the standard limb-sparing procedure was not affected, (2) demonstrate the animal's recovery and welfare are not affected compared with the expected outcomes of the standard limb-sparing procedure, and (3) demonstrate that the fragile bioactive scaffold remains intact through the normal recovery and healing process. In each of the patients operated on to date, immediate weight-bearing and mobility was demonstrated for the patient under the standard care regimen.

FIG. 6 shows a radiograph 600 depicting an example device with sleeve 410, fixation member 440, and scaffold 430. At five months post-op, as shown in the radiograph 600 in FIG. 6, scaffolds were wholly intact, and patients presented normal gait, excellent limb function and normal, positive behavioral attitude. Note the excellent detail in scaffold structure, which was produced in segments to enable visualization of relative movement between segments (there was none apparent). Scaffolds up to 4 inches in length can be fabricated. At eight-months post-op, the scaffolds showed no signs of damage and patients continued to demonstrate that the devices and systems described herein facilitate normal function and quality of life. The study progressed for each client-owned dog until the dog passed away for any reason, at which time the treated limb was received for further evaluation, including stiffness testing, CT, and histological/histomorphometric/histopathologic evaluation. The study validates the endoprosthetic devices, systems, and methods described herein.

The sleeve design (as shown in FIG. 4) can be modified to exert 0.01 mm of transverse compression to the scaffold under ION of loading, increasing linearly to 0.1 mm at 100N of loading. In this disclosure, the design may be adjusted to induce transverse micromotion that exerts strain greater than 7%, which are shown to exert a positive influence on callus formation, a key step in the bone healing process. These design goals will be applied to sleeves in the proposed study prior to implementation.

Although specific embodiments of the disclosure have been described, one of ordinary skill in the art will recognize that numerous other modifications and alternative embodiments are within the scope of the disclosure. Further, while various illustrative implementations have been described in accordance with embodiments of the disclosure, one of ordinary skill in the art will appreciate that numerous other modifications to the illustrative implementations described herein are also within the scope of this disclosure.

Although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments do not include, certain features, elements, and/or steps. Thus, such conditional language is not generally intended to imply that features, elements, and/or steps are in any way required for one or more embodiments.

What is claimed is:

1. A method for treating a bone defect of a bone having a first bone structure and a second bone structure, the method comprising:
positioning a biodegradable scaffold within a passage of a biodegradable sleeve, wherein the passage extends from a first end to a second end of the biodegradable sleeve, wherein a geometry of the biodegradable sleeve induces strain on a portion of the biodegradable scaffold when a force is applied to the biodegradable sleeve; and
positioning the biodegradable sleeve along the first bone structure and the second bone structure to span the bone defect.

2. The method of claim 1, wherein the biodegradable sleeve includes a first sleeve portion and a second sleeve portion, wherein the first sleeve portion has a greater rigidity than the second sleeve portion.

3. The method of claim 2, wherein the first sleeve portion includes a material that is not included in the second sleeve portion.

4. The method of claim 2, wherein the first sleeve portion has a larger wall thickness than the second sleeve portion as measured from an outer surface of the sleeve to an inner surface of the sleeve.

5. The method of claim 4, wherein the second sleeve portion includes a concavity defined by the outer surface of the sleeve.

6. The method of claim 4, wherein the first sleeve portion includes one or more ribs extending outwardly from the outer surface of the sleeve.

7. The method of claim 1, further comprising resecting a region of bone between the first bone structure and the second bone structure and encompassing the bone defect before positioning the biodegradable scaffold within the passage of the biodegradable sleeve.

8. The method of claim 1, wherein the biodegradable scaffold comprises a biodegradable osteogenic scaffold.

9. The method of claim 1, further comprising coupling the biodegradable sleeve to a fixation member before positioning the biodegradable sleeve and attaching the fixation member to each of the first bone structure and the second bone structure after positioning the biodegradable sleeve.

10. The method of claim 9, wherein coupling the biodegradable sleeve to the fixation member comprises sliding the biodegradable sleeve onto the fixation member.

11. The method of claim 9, wherein attaching the fixation member to each of the first bone structure and the second bone structure comprises:
attaching the fixation member to the first bone structure using a first plurality of screws; and
attaching the fixation member to the second bone structure using a second plurality of screws.

12. The method of claim 9, further comprising treating the fixation member with an antibiotic to inhibit infection.

13. The method of claim 9, further comprising detaching the fixation member from the first bone structure and the second bone structure and removing the fixation member from the patient after the bone defect has healed and the biodegradable scaffold and the biodegradable sleeve have degraded.

14. The method of claim 1, further comprising treating the biodegradable scaffold with one or more bioactive agents to inhibit infection and/or facilitate osteoinduction, osteoconduction, osteointegration, vascularization, or angiogenesis.

15. The method of claim 1, further comprising treating the biodegradable sleeve with an antibiotic to inhibit infection.

16. The method of claim 1, wherein loading of the first bone structure or second bone structure causes the application of force to the biodegradable sleeve.

17. The method of claim 16, wherein the first bone structure or second bone structure remain static relative to the biodegradable sleeve during loading of the first bone structure or second bone structure.

18. The method of claim 1, wherein positioning the biodegradable sleeve along the first bone structure and the second bone structure comprises:
inserting an end portion of the first bone structure into the first end of the biodegradable sleeve such that the end portion of the first bone structure contacts the biodegradable scaffold; and
inserting an end portion of the second bone structure into the second end of the biodegradable sleeve such that the end portion of the second bone structure contacts the biodegradable scaffold.

19. The method of claim 1, further comprising:
obtaining computed tomography scans of the patient; and
fabricating the biodegradable scaffold and the biodegradable sleeve based at least in part on the computed tomography scans.

20. The method of claim 19, further comprising fabricating a cutting guide based at least in part on the computed tomography scans, wherein resecting the region of bone between the first bone structure and the second bone structure and encompassing the bone defect comprises resecting the region of bone using the cutting guide.

21. The method of claim 1, wherein the biodegradable scaffold is formed of a calcium phosphate-based material or a composite comprising a calcium phosphate and a polymer, and wherein the biodegradable sleeve is formed of polycaprolactone or a composite comprising a calcium phosphate and polycaprolactone.

22. A device for treating a bone defect of a bone having a first bone structure and a second bone structure, the device comprising:
a biodegradable scaffold;
a biodegradable sleeve configured for positioning along the first bone structure and the second bone structure to span the bone defect, wherein the biodegradable sleeve defines a passage configured for accepting the biodegradable scaffold, wherein the passage extends from a first end to a second end of the biodegradable sleeve, wherein a geometry of the biodegradable sleeve induces strain on a portion of the biodegradable scaffold when a force is applied to the biodegradable sleeve.

23. The device of claim 22, wherein the biodegradable sleeve includes a first sleeve portion and a second sleeve portion, wherein the first sleeve portion has a greater rigidity than the second sleeve portion.

24. The device of claim 23, wherein the first sleeve portion includes a material that is not included in the second sleeve portion.

25. The device of claim 23, wherein the first sleeve portion has a larger wall thickness than the second sleeve portion as measured from an outer surface of the sleeve to an inner surface of the sleeve.

26. The device of claim 25, wherein the second sleeve portion includes a concavity defined by the outer surface of the sleeve.

27. The device of claim 25, wherein the first sleeve portion includes one or more ribs extending outwardly from the outer surface of the sleeve.

28. The device of claim 22, wherein the biodegradable scaffold comprises a biodegradable osteogenic scaffold.

29. The device of claim 22, further comprising a fixation member configured for coupling to the biodegradable sleeve and attaching to each of the first bone structure and the second bone structure.

30. The device of claim 29, wherein the passage is a first passage, the first passage extending from the first end to the second end of the biodegradable sleeve, wherein the biodegradable sleeve comprises:
a second passage extending from the first end to the second end of the biodegradable sleeve and configured for receiving a portion of the fixation member therein.

31. The device of claim 30, wherein the biodegradable sleeve further comprises support ribs disposed between the first passage and the second passage and extending from the first end to the second end of the biodegradable sleeve, and wherein the first passage is in communication with the second passage through a gap defined between the support ribs.

32. The device of claim 31, wherein a portion of the biodegradable scaffold extends into the gap when the biodegradable scaffold is positioned within the biodegradable sleeve.

33. The device of claim 30, wherein the first passage is further configured for receiving an end portion of the first bone structure and an end portion of the second bone structure therein.

34. The device of claim 30, wherein the biodegradable sleeve further comprises a plurality of apertures disposed along one or more sides of the biodegradable sleeve and extending from an outer surface of the biodegradable sleeve to the first passage, and wherein the apertures are configured for controlling transcortical perfusion.

35. The device of claim 30, wherein the biodegradable sleeve further comprises a plurality of openings disposed along a side of the biodegradable sleeve and extending from an outer surface of the biodegradable sleeve to the second passage, and wherein the openings expose respective portions of the fixation member when the fixation member is coupled to the biodegradable sleeve and are configured for receiving respective collagen sponges carrying an antibiotic therein.

36. The device of claim 30, wherein the biodegradable scaffold comprises a scaffold passage extending from a first end to a second end of the biodegradable scaffold and configured for receiving a collagen sponge carrying bioactive agents.

37. The device of claim 30, wherein the fixation member comprises:
a first portion configured for extending beyond the first end of the biodegradable sleeve when the fixation member is coupled to the biodegradable sleeve;
a second portion configured for extending beyond the second end of the biodegradable sleeve when the fixation member is coupled to the biodegradable sleeve; and
an intermediate portion configured for positioning within the second passage when the fixation member is coupled to the biodegradable sleeve.

38. The device of claim 30, wherein the first end of the passage defines a first periosteum slot and the second end of the passage defines a second periosteum slot, wherein the first periosteum slot is configured for insertion of an end portion of periosteum associated with the first bone structure and the second periosteum slot is configured for insertion of an end portion of periosteum associated with the second bone structure.

39. The device of claim 22, wherein loading of the first bone structure or second bone structure causes the application of force to the biodegradable sleeve.

40. The device of claim 39, wherein the first bone structure or second bone structure remain static relative to the biodegradable sleeve during loading of the first bone structure or second bone structure.

41. The device of claim 22, wherein the biodegradable scaffold is formed of a calcium phosphate-based material or a composite comprising a calcium phosphate and a polymer, and wherein the biodegradable sleeve is formed of polycaprolactone or a composite comprising a calcium phosphate and polycaprolactone.
